(19) United States
(12) Patent Application Publication
(10) Pub. No.: US 2013/0041005 A1
(43) Pub. Date: Feb. 14, 2013

(54) PHARMACEUTICAL COMPOSITIONS COMPRISING NEUROPILIN INHIBITORS, AND THEIR USE FOR THE PREVENTION AND/OR TREATMENT OF ANGIOGENIC DISORDERS AND CANCERS

(71) Applicant: TRAGEX PHARMA, Paris (FR)

(72) Inventors: Reda HADJ-SLIMANE, PARIS (FR); Christiane GARBAY, PARIS (FR)

(73) Assignee: TRAGEX PHARMA, PARIS (FR)

(21) Appl. No.: 13/655,870

(22) Filed: Oct. 19, 2012

Related U.S. Application Data
(63) Continuation-in-part of application No. PCT/EP2012/058721, filed on May 11, 2012.

(30) Foreign Application Priority Data
May 13, 2011 (EP) 11166117.9

(51) Int. Cl. A61K 31/4184; A61P 35/00; A61P 35/02; A61P 9/00; A61P 17/02; A61P 1/00; A61P 11/00; A61P 17/00; A61P 37/00; A61P 29/00; C07D 405/12; A61P 27/02

(52) U.S. Cl. 514/394; 548/305.1

(57) ABSTRACT

The present invention relates to a compound of general formula (I),

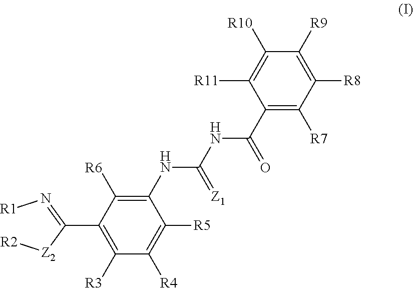

and to a pharmaceutical composition comprising a compound of general formula (I) or esters or salts thereof, in association with at least one pharmaceutically acceptable vehicle; and to the use thereof for inhibiting the Neuropilin pathways in the treatment of cancer and of angiogenic diseases.

### FIELD OF INVENTION

The present invention relates to the field of the treatment and/or prevention of diseases, disorders or conditions related to cancers and/or angiogenesis. The present invention particularly relates to specific chemical compounds, herein shown to be inhibitors of the Neuropilin pathways, and to the pharmaceutical use thereof, especially for treating and/or preventing diseases, disorders or conditions related to cancers and/or angiogenesis.

### BACKGROUND OF INVENTION

Neuropilin is a transmembrane glycoprotein acting as a co-receptor for two types of ligands: members of the semaphorin family, a family of axon guidance modulators, and members of the vascular endothelial growth factor (VEGF) family. Neuropilin exists in two forms, namely Neuropilin-1 (NRP-1) and 2 (NRP-2). NRP-1 and NRP-2 present different affinity and specificity of ligands. Both proteins may function as co-receptors binding an extracellular ligand with high affinity, and complexing with other transmembrane receptors of the same ligand (e.g. VEGF-R2 for VEGF), to modulate signal transduction within the cell.

The overexpression of NRP-1 and NRP-2 has been demonstrated to be correlated with tumor progression and patient prognosis in specific tumor types. Moreover, Neuropilin seems to contribute to tumor formation in a number of diseases such as prostate, breast and colon cancer (Ellis L M, Mol Cancer Ther, 2006, 5:1099-107). Neuropilin may mediate the effects of VEGF and semaphorins on the proliferation, survival and migration of cancer cells.

Neuropilin may thus be a promising target for preventing and/or treating diseases, disorders or conditions related to cancer.

Moreover, Neuropilin is involved, as a member of the VEGF pathway, in the process of angiogenesis. Angiogenesis is a fundamental process of new blood vessels formation by endothelial cells. This formation involves three different steps, (i) the migration, (ii) the growth and (iii) the differentiation of endothelial cells. Disturbance of this tightly regulated phenomenon may lead to several diseases, wherein tissues and organs are subjected to the invasion by neovessels.

Therefore, an inhibitor of Neuropilin may also be useful for the prevention and/or the treatment of angiogenic diseases, disorders or conditions.

Several means for inhibiting Neuropilin have been described in the art. For example, WO03/035100 describes a modulator of the expression or activity of Neuropilin. WO2007/056470 describes antibodies against NRP-1. U.S. Pat. No. 7,335,357 describes proteins or protein fragments, especially members of the semaphorin collapsin family, as inhibitors of Neuropilin. WO2009/099959 and WO99/55855 describe nucleic acids, such as, for example, antisens oligonucleotides, for modulating the expression of Neuropilin genes. Jarvis et al. (J. Med. Chem. 2010, 53, 2215-26) describes particular chemical compounds (such as, for example, EG00229) for inhibiting the interaction between NRP-1 and VEGF-A.

However, to the Applicant knowledge, there are no clinical trials or current medical development susceptible to lead to a medicament, involving a small molecule inhibiting Neuropilin.

Therefore, due to the great potential of such compounds for preventing and/or treating diseases, disorders or conditions related to cancers and/or angiogenesis, there is still a need for a small molecule, such as, for example, a chemical compound, for inhibiting the Neuropilin pathway.

This invention relates to chemical compounds of general formula (I),

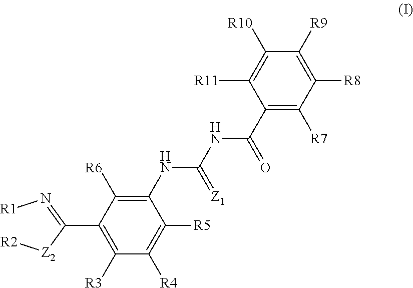

wherein
Z1 is S or O, preferably S;
—Z2- is —NR^{a}—, wherein R^{a} is H, OH, alkyl, alkoxy, aryle, alkenyl, alkynyl, amine, preferably R^{a} is H; —N═; —O—; —S—; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—; preferably —Z2- is —NH— or —N═;
each of R1 and R2 is independently H, OH, alkyl, alkoxy, aryl, alkenyl, alkynyl, amine, NO₂, Cl, Br, I, F; preferably R1 and R2 together and with N and Z2 form an heterocycle eventually substituted, more preferably a substituted azocine, 3H-indole, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, or a substituted imidazole and even more preferably a benzimidazole;
R5 is H, OH, C₁₋₄ alkyl, C₁₋₄ alkoxy, C₁₋₄alkenyl, C₁₋₄alkynyl, amine, NO₂, Cl, F, Br, I; more preferably R5 is H or CH₃; even more preferably R5 is CH₃;
each of R8 and R9 is independently, H, OH, alkyl, alkoxy, aryl, alkenyl, alkynyl, amine, NO₂, Cl, Br, I, F; preferably R8 and R9 together form a cycle comprising 5 or 6 atoms, preferably an heterocycle comprising 5 or 6 atoms, more preferably a 1,3-dioxacyclopentene or a 1,4-dioxane; and
each of R3, R4, R6, R7, R10 and R11 is independently, H, OH, C₁₋₄ alkyl, C₁₋₄ alkoxy, C₁₋₄alkenyl, C₁₋₄alkynyl, amine, NO₂, F, Cl, Br, I; preferably H, CH₃, OCH₃, OH, NH₂, NO₂, Cl, F, Br, I; more preferably is H or CH₃; even more preferably is H.
   The present invention also relates to pharmaceutical compositions comprising a compound of general formula (I) or esters or salts thereof, in association with at least one pharmaceutically acceptable vehicle.

After having found the compounds of the invention and their interest as Neuropilin inhibitors, the Applicant got aware of WO2009/130422 relating to chemical compounds for inhibiting the Hedgehog pathways. The experiments of WO2009/130422 are carried out exclusively on embryonic cell lines. The results of these experiments showed that the chemical compounds of WO2009/130422 inhibited the differentiation of embryonic cell lines (embryonic mouse cells of the adipocytes lineage C3H10T1/2 and human embryonic kidney cells, HEK293).

WO2009/130422 does not give any teaching on the NRP pathways. Even though Neuropilin may be involved in embryonic development, it is generally recognized that Neuropilin impacts processes of embryonic endothelial cells or nerve cells only (vessel formation and nerve fiber guidance). And WO2009/130422 gives information on adipocytes and kidney cell lines differentiation exclusively.

Moreover, the effect of the compounds of WO2009/130422 on the differentiation of embryonic cells, which are not cancer cells or endothelial cells, does not provide any evidence that these compounds may be useful in preventing and/or treating diseases, disorders or conditions related to cancers and/or angiogenesis.

The Inventors herein show that the chemical compounds of the invention inhibit the interaction between Neuropilin and the components of the VEGF pathway. Without willing to be bound to a theory, the Inventors suggest that the chemical compounds of the invention physically interact with the Neuropilin molecule, and thus block the associated signaling pathways, thereby providing beneficial effect for preventing and/or treating diseases associated to the Neuropilin pathways, such as, for example, cancers (e.g. solid tumor or metastasis) or diseases associated with abnormal angiogenesis (e.g. atherosclerosis, proliferative retinopathies, or rheumatoid arthritis).

### DEFINITIONS

“Subject”: an animal, including a human. In the sense of the present invention, a subject may be a patient, i.e. a person receiving medical attention, undergoing or having underwent a medical treatment, or monitored for the development of a disease.
“Treating a disease”: refers to both therapeutic treatment and prophylactic or preventative measures, wherein the object is to prevent or slow down (lessen) the targeted pathologic condition or disorder. Treating a disease may thus relates to reducing, alleviating or avoiding the occurrence at least one adverse effect or symptom of a disease, disorder or condition associated with a deficiency in an organ, tissue or cell function. Treating a disease thus includes “Preventing a disease”, wherein this expression refers to preventing or avoiding the occurrence of symptom. In the present invention, the term “prevention” may refer to a secondary prevention, i.e. to the prevention of the re-occurrence of a symptom or a relapse of the disease. It may also refer, when the disease is cancer, to the occurrence of metastases after the treatment and/or the removal of a tumor.
“Therapeutically effective amount”: refers to the amount of a therapeutic agent necessary and sufficient for preventing a disease; slowing down or stopping the progression, aggravation, or deterioration of one or more symptoms of the disease or condition; alleviating the symptoms of the disease or condition; curing or treating the disease or condition.
“Alkyl”: means any saturated linear or branched hydrocarbon chain, with 1 to 12 carbon atoms, preferably 1 to 6 carbon atoms, and more preferably methyl, ethyl, propyl, isopropyl, n-butyl, sec-butyl, isobutyl and tert-butyl.
“Alkenyl”: means any linear or branched hydrocarbon chain having at least one double bond, of 2 to 12 carbon atoms, and preferably 2 to 6 carbon atoms.
“Alkynyl”: means any linear or branched hydrocarbon chain having at least one triple bond, of 2 to 12 carbon atoms, and preferably 2 to 6 carbon atoms.
“Amine”: means any compound derived from ammoniac NH₃ by substitution of one or more hydrogen atoms with an organic radical, preferably in the invention, the term amine stands for NH₂ or NHR, wherein R is an alkyl, preferably a C₁₋₄ alkyl.
“Aryl”: means a mono- or polycyclic system of 5 to 20, and preferably 6 to 12, carbon atoms having one or more aromatic rings (when there are two rings, it is called a biaryl) among which it is possible to cite the phenyl group, the biphenyl group, the 1-naphthyl group, the 2-naphthyl group, the tetrahydronaphthyl group, the indanyl group and the binaphthyl group. The term aryl also means any aromatic ring including at least one heteroatom chosen from an oxygen, nitrogen or sulfur atom. The aryl group can be substituted by 1 to 3 substituents chosen independently of one another, among a hydroxyl group, a linear or branched alkyl group comprising 1, 2, 3, 4, 5 or 6 carbon atoms, in particular methyl, ethyl, propyl, butyl, an alkoxy group or a halogen atom, in particular bromine, chlorine and iodine.
“Alkoxy”: refers to any O-alkyl or O-aryl group.
“Heterocycle”: means a ring with 5 or 6 links containing 1 to 2 heteroatoms chosen from O, S, N, optionally substituted with an alkyl.
“About” preceding a figure means plus or less 10% of the value of said figure.

### DETAILED DESCRIPTION

The present invention relates to a compound of general formula (I):

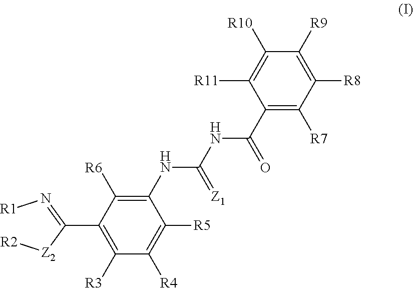

wherein:
Z1 is S or O, preferably S;
—Z2- is —NR^{a}—, wherein R^{a} is H, OH, alkyl, alkoxy, aryl, alkenyl, alkynyl, amine, preferably H; —N═; —O—; —S—; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—, preferably —Z2- is —NH— or —N═;
each of R1 and R2 independently is H, OH, alkyl, alkoxy, aryle, alkenyl, alkynyl, amine, NO₂, Cl, Br, I, F; preferably R1 and R2 together and with N and Z2 form an heterocycle eventually substituted, more preferably a substituted azocine, 3H-indole, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, or a substituted imidazole and even more preferably a benzimidazole;
R5 is H, OH, C₁₋₄ alkyl, C₁₋₄ alkoxy, C₁₋₄alkenyl, C₁₋₄alkynyl, amine, NO₂, F, Cl, Br, I; preferably R5 is H, CH₃, OCH₃, OH, NH₂, NO₂, Cl, F, Br, I; more preferably R5 is H or CH₃; even more preferably R5 is CH₃;
each of R8 and R9 independently is H, OH, alkyl, alkoxy, aryl, alkenyl, alkynyl, amine, NO₂, Cl, Br, I, F; preferably R8 and R9 together form a cycle comprising 5 or 6 atoms, preferably an heterocycle comprising 5 or 6 atoms, more preferably a 1,3-dioxacyclopentene or a 1,4-dioxane; and
each of R3, R4, R6, R7, R10 and R11 independently is H, OH, C₁₋₄ alkyl, C₁₋₄ alkoxy, C₁₋₄alkenyl, C₁₋₄alkynyl, amine, preferably NH₂, NO₂, F, Cl, Br, I; preferably H, CH₃, OCH₃, OH, NH₂, NO₂, Cl, F, Br, I; more preferably is H or CH₃; even more preferably is H.
   In one embodiment, the heterocycle formed by R1, R2, N and Z2 is not an indole.
   In one embodiment of the invention, said heterocycle is selected from the group consisting of a substituted azocine, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, and a substituted imidazole.
   In one embodiment of the invention, said heterocycle is a benzimidazole.
   In one embodiment of the invention, —Z2- is —NR^{a}—, wherein R^{a} is H, OH, alkyl, alkoxy, aryl, alkenyl, alkynyl, amine, preferably H; —N═; —O—; —S—; preferably —Z2- is —NH— or —N═.

According to one embodiment, the compound of general formula (I) of the invention is of general formula (I) wherein
Z1 is S or O, preferably S;
—Z2- is —NR^{a}—, wherein R^{a} is H, OH, alkyl, alkoxy, aryl, alkenyl, alkynyl, amine, preferably H; —N═; —O—; —S—; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—, preferably —Z2- is —NH— or —N═;
R1 and R2 together and with N and Z2 form an heterocycle eventually substituted, more preferably a substituted azocine, 3H-indole, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, or a substituted imidazole and even more preferably a benzimidazole;
R⁵ is H or C₁₋₄ alkyl;
R8 and R9 together form a cycle comprising 5 or 6 atoms, preferably an heterocycle comprising 5 or 6 atoms, more preferably a 1,3-dioxacyclopentene or a 1,4-dioxane;
each of R3, R4, R6, R7, R10 and R11 independently is H or C₁₋₄ alkyl.
   In one embodiment, the heterocycle formed by R1, R2, N and Z2 is not an indole.

In one embodiment of the invention, said heterocycle is selected from the group consisting of a substituted azocine, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, and a substituted imidazole.

In one embodiment of the invention, said heterocycle is a benzimidazole.
In one embodiment of the invention, —Z2- is —NR^{a}—, wherein R^{a} is H, OH, alkyl, alkoxy, aryl, alkenyl, alkynyl, amine, preferably H; —N═; —O—; —S—; preferably —Z2- is —NH— or —N═.

According to one embodiment, the compound of general formula (I) of the invention is of general formula (I) wherein
Z1 is S;
—Z2- is —NH— or —N═ or —S—;
R1 and R2 together and with N and Z2 form a substituted imidazole and more preferably a benzimidazole;
R5 is H or CH₃ or C₂H₅;
R8 and R9 together form a cycle comprising 5 or 6 atoms, preferably an heterocycle comprising 5 or 6 atoms, more preferably a 1,3-dioxacyclopentene or a 1,4-dioxane;
R3, R4, R6, R7, R10 and R11 are H.

According to one embodiment, the compound of formula (I) of the invention has the general formula (Ia):

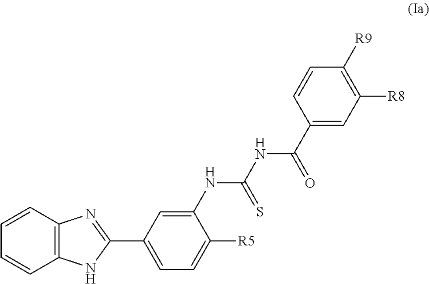

wherein

R5 is H or CH₃;

R8 and R9 together form a 1,3-dioxacyclopentene or a 1,4-dioxane.

Preferably, the compound of formula (I) of the invention has the general formula (Ia) wherein

—R⁵ is H or CH₃;

R⁸ and R⁹ together form a 1,4-dioxane.

Preferred compounds of formula (I) include:
N-[5-(1H-benzimidazol-2-yl)-2-methylphenyl]-N′-(2,3-dihydro-1,4-benzodioxin-6-ylcarbonyl)thiourea (compound 1)

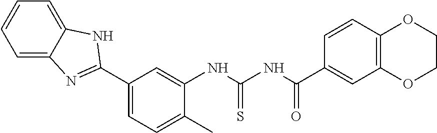 [SMILES: Cc1ccc(-c2nc3ccccc3[nH]2)cc1NC(=S)NC(=O)c1ccc2c(c1)OCCO2]

N-[3-(1H-benzimidazol-2-yl)phenyl]-N′-(2,3-dihydro-1,4-benzodioxin-6-ylcarbonyl)thiourea (compound 2):

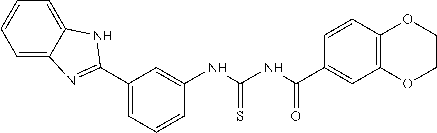 [SMILES: O=C(NC(=S)Nc1cccc(-c2nc3ccccc3[nH]2)c1)c1ccc2c(c1)OCCO2]

N-[3-(1H-benzimidazol-2-yl)phenyl]-N′-(1,3-benzodioxol-5-ylcarbonyl)thiourea (compound 3)

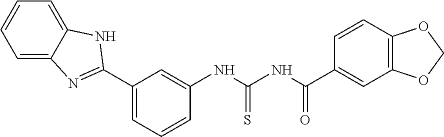 [SMILES: O=C(NC(=S)Nc1cccc(-c2nc3ccccc3[nH]2)c1)c1ccc2c(c1)OCO2]

Below are some of the chemical characteristics of the compounds 1 and 2 of the invention.

| | Compound 1 | Compound 2 |
|---|---|---|
| Molecular formula | C₂₄H₂₀N₄O₃S | C₂₃H₁₇N₄O₃S |
| State | Powder | Powder |
| Colour | Yellow | Yellow |
| Molecular weight <500 | 444 g/mol | 430 g/mol |
| H-bond donors <5 | 4 | 3 |
| H-bond acceptor <10 | 3 | 5 |
| logP <5 | 3.15 | 2.94 |
| Melting point | 202 | 212 |

The present invention also relates to a pharmaceutical composition comprising a compound of general formula (I) as hereinabove described, or esters or salts thereof, in combination with at least one pharmaceutically acceptable vehicle.

The present invention thus relates to a pharmaceutical composition comprising a compound of general formula (I):

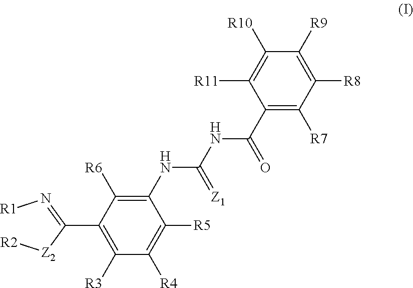

wherein:
Z1 is S or O, preferably S;
—Z2- is —NR^{a}—, wherein R^{a} is H, OH, alkyl, alkoxy, aryl, alkenyl, alkynyl, amine, preferably H; —N═; —O—; —S—; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—, preferably —Z2- is —NH— or —N═;
each of R1 and R2 is independently H, OH, alkyl, alkoxy, aryle, alkenyl, alkynyl, amine, NO₂, Cl, Br, I, F; preferably R1 and R2 together and with N and Z2 form an heterocycle eventually substituted, more preferably a substituted azocine, 3H-indole, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, or a substituted imidazole and even more preferably a benzimidazole;
R5 is H, OH, C₁₋₄ alkyl, C₁₋₄ alkoxy, C₁₋₄alkenyl, C₁₋₄alkynyl, amine, NO₂, F, Cl, Br, I; preferably R5 is H, CH₃, OCH₃, OH, NH₂, NO₂, Cl, F, Br, I; more preferably R5 is H or CH₃; even more preferably R5 is CH₃;
each of R8 and R9 is independently H, OH, alkyl, alkoxy, aryl, alkenyl, alkynyl, amine, NO₂, Cl, Br, I, F; preferably R8 and R9 together form a cycle comprising 5 or 6 atoms, preferably an heterocycle comprising 5 or 6 atoms, more preferably a 1,3-dioxacyclopentene or a 1,4-dioxane; and
each of R3, R4, R6, R7, R10 and R11 is independently H, OH, C₁₋₄ alkyl, C₁₋₄ alkoxy, C₁₋₄alkenyl, C₁₋₄alkynyl, amine, preferably NH₂, NO₂, F, Cl, Br, I; preferably H, CH₃, OCH₃, OH, NH₂, NO₂, Cl, F, Br, I; more preferably is H or CH₃; even more preferably is H;
   or esters or salts thereof; in association with at least one pharmaceutically acceptable vehicle.
   In one embodiment, the heterocycle formed by R1, R2, N and Z2 is not an indole.
   In one embodiment of the invention, said heterocycle is selected from the group consisting of a substituted azocine, indazole, 2-imidazoline, 2-pyrazoline, benzothiazole, purine, pyrimidine, pyridine, pyridazine, pyrazine, pyrazole, thiazole, isothiazole, oxazole, isoxazole, quinoline, isoquinoline, quinoxaline, quinazoline, 1-8-naphthyridine, perimidine, [1,10]-phenantroline, phthalazine, pteridine, triazole, triazine, furazan, 6H-1,2,5-thiadiazine, 1,3,4-thiadiazole, tetrazole, and a substituted imidazole.
   In one embodiment of the invention, said heterocycle is a benzimidazole.
   In one embodiment of the invention, —Z2- is —NR^{a}—, wherein R^{a} is H, OH, alkyl, alkoxy, aryle, alkenyl, alkynyl, amine, preferably H; —N═; —O—; —S—; preferably —Z2- is —NH— or —N═.

In one embodiment, the pharmaceutical composition of the invention comprises several compounds of general formula (I) or esters or salts thereof. Accordingly, the pharmaceutical composition of the invention may comprise a mixture of compounds of general formula (I) or esters or salts thereof. In one embodiment, the composition of the invention comprises the compound 1 and the compound 2 as hereinabove described. In one embodiment, the composition of the invention comprises the compound 1 and the compound 3 as hereinabove described. In one embodiment, the composition of the invention comprises the compound 2 and the compound 3 as hereinabove described. In one embodiment, the composition of the invention comprises the compound 1, the compound 2 and the compound 3 as hereinabove described.

The present invention also relates to a medicament comprising the compound of the invention as hereinabove described.

The present invention also relates to a medicament comprising the pharmaceutical composition as hereinabove described.

This invention also relates to a method for manufacturing compounds or compositions as hereinabove described, wherein a compound of general formula (II)

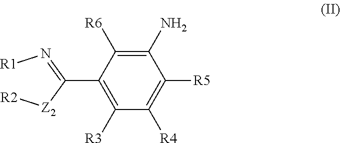

wherein R1, R2, Z2, R3, R4, R5 and R6 are as described hereinabove,
is reacted with a compound of general formula (IV)

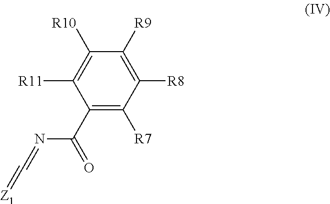

in a refluxing solvent, such as, for example, acetone, to give compounds of general formula (I).

According to an embodiment, the compound of general formula (IV) is obtained by reacting a compound of general formula (III)

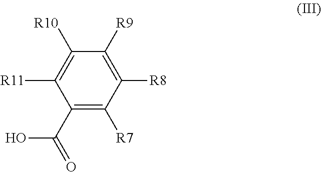

with (i) SOCl₂ and (ii) NH₄Z1CN, wherein Z1 is as hereinabove defined, in the presence of a base, such as, for example, K₂CO₃ in a refluxing solvent, such as, for example, acetone.

The reaction process is shown below.

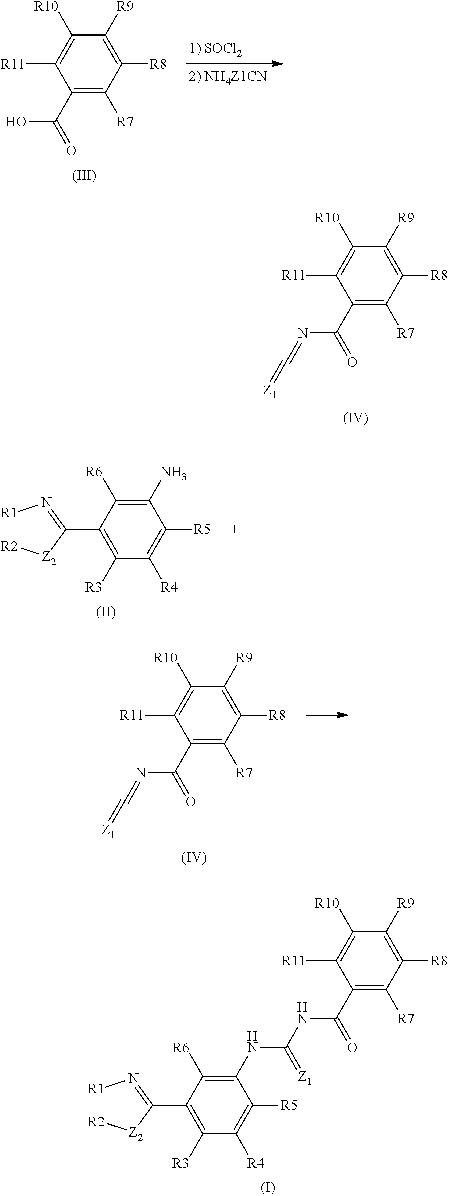

The reaction process for synthetizing the compound 1 of the invention is shown below:

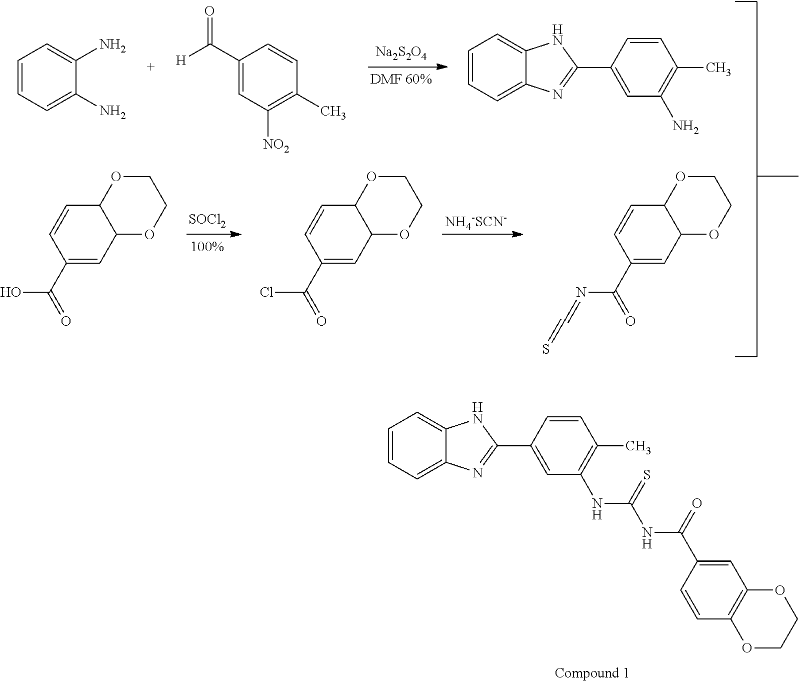

The invention also relates to a compound or a pharmaceutical composition as hereinabove described for inhibiting the Neuropilin pathways.

The invention also relates to a method for inhibiting the Neuropilin pathways in a subject in need thereof, comprising administering to said subject a therapeutically effective amount of a compound of general formula (I) or of a pharmaceutical composition comprising a compound of general formula (I).

The invention also relates to an inhibitor of the Neuropilin pathways, which is a pharmaceutical composition or a compound of general formula (I) as hereinabove described. Surprisingly, the Inventors showed that the compounds of the invention inhibited the VEGF-NRP-1 binding as well as the VEGF-NRP-2 binding (see Examples). Without willing to be bound to a theory, the Inventors suggest that the chemical compounds of the invention physically interact with the Neuropilin molecule, and thus block the associated signaling pathways.

In one embodiment of the invention, the compound of general formula (I) inhibits the binding of VEGF to NRP-1, preferably with an IC₅₀ of less than or equal to about 45 μM, more preferably about 38 μM, and even more preferably less than or equal to about 34 μM.

In another embodiment, the compound of general formula (I) inhibits the binding of VEGF to NRP-2, preferably with an IC₅₀ of less than or equal to about 50 μM, more preferably about 42 μM, and even more preferably less than or equal to about 35 μM.

As used herein, the term “IC₅₀” represents the concentration of a compound that is required for 50% inhibition in vitro.

Methods for measuring IC₅₀ are well-known by the skilled artisan. Examples of such methods include, but are not limited to, ELISA and sandwich ELISA.

The present invention relates to a compound of general formula (I) for use in the treatment of a cancer in a subject in need thereof.

Accordingly, the present invention also relates to a pharmaceutical composition comprising a therapeutically effective amount of a compound of general formula (I) in association with a pharmaceutically acceptable vehicle, for use in the treatment of a cancer in a subject in need thereof.

The Inventors surprisingly showed that the compounds of the invention inhibited cancer cells proliferation and induced cancer cells apoptosis (FIG. 5 and Tables 2-3), were cytotoxic for cancer cell lines of different origins (FIGS. 1 and 4; Table 2), and that they inhibited the adhesion of cancer cells (FIGS. 1A to 1C). Moreover, the Inventors demonstrated that the compounds of the invention were efficient in vivo as anti-cancer agents (FIG. 8). Indeed, the administration to mice of the compounds of the invention inhibited the expansion of a tumor, and resulted in a prolonged survival of the mice.

In one embodiment, the compound or the pharmaceutical composition of the invention may be used for inhibiting the proliferation of cancer cells. Preferably, said inhibition has an IC₅₀ of less than or equal to about 1 μM, preferably less than or equal to about 0.6, more preferably less than or equal to about 0.4.

In one embodiment of the invention, the compound or the pharmaceutical composition of the invention may be used for inducing the apoptosis of cancer cells.

In one embodiment, the compound or the pharmaceutical composition of the invention may be used for inhibiting the growth of a tumor. In one embodiment, the compound or the pharmaceutical composition of the invention may be used for inhibiting the number and the development of cancerous cells within the body of the subject.

In one embodiment, the compound or the pharmaceutical composition of the invention may be used for inhibiting the occurrence of metastases.

According to an embodiment, cancer is solid tumor, metastatic cancer or non-metastatic cancer.

In one embodiment, the cancer may originated in the bladder, blood, bone, bone marrow, brain, breast, cervic area, colon, esophagus, eye and periocular including subconjunctival tissues, duodenum, small intestine, large intestine, rectum, anus, gum, head, kidney, liver, lung, nasopharynx, neck, ovary, pancreas, prostate, skin, stomach, testis, tongue, or uterus.

Examples of cancer include, but are not limited to, carcinoma, adenocarcinoma, lymphoma, blastoma, hepatoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, fibrosarcoma, Burkitt's lymphoma, hepatocellular cancer, gastric or stomach cancer (including gastrointestinal cancer), pancreatic cancer, such as, for example, pancreatic carcinoma, glioblastoma, cervical cancer, ovarian cancer, liver cancer, bladder cancer, hepatoma, breast cancer, colon cancer, such as, for example, colon adenocarcinoma (including a colon adenocarcinoma grade II), colorectal cancer, such as, for example, colorectal carcinoma, endometrial or uterine carcinoma, salivary gland carcinoma, kidney or renal cancer, liver cancer, prostate cancer, such as, for example, prostate adenocarcinoma, vulval cancer, thyroid cancer, osteosarcoma, neuroblastoma, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

According to an embodiment, the cancer is breast cancer, cervical cancer, pancreatic carcinoma, colorectal carcinoma, colon adenocarcinoma, such as, for example, colon adenocarcinoma grade II, osteosarcoma, prostate cancer, such as, for example, prostate adenocarcinoma, gioblastoma or neuroblastoma.

According to an embodiment, the compound or the pharmaceutical composition of the invention is injected, preferably systemically injected. Examples of formulations adapted to systemic injections include, but are not limited to, liquid solutions or suspensions, solid forms suitable for solution in, or suspension in, liquid prior to injection. Examples of systemic injections include, but are not limited to, intravenous, subcutaneous, intramuscular, intradermal and intraperitoneal injection, and perfusion.

According to another embodiment, the compound or the composition of the invention is intratumorally or intralesionally injected.

According to an embodiment, when injected, the compound or the pharmaceutical composition of the invention is sterile. Methods for obtaining a sterile pharmaceutical composition include, but are not limited to, GMP synthesis (GMP stands for “Good manufacturing practice”).

According to another embodiment, the compound or the pharmaceutical composition of the invention is orally administered. Examples of formulations adapted to oral administration include, but are not limited to, solid forms, liquid forms and gels. Examples of solid forms adapted to oral administration include, but are not limited to, pill, tablet, capsule, soft gelatin capsule, hard gelatin capsule, caplet, compressed tablet, cachet, wafer, sugar-coated pill, sugar coated tablet, orodispersing/orodisintegrating tablet, powder, solid forms suitable for solution in, or suspension in, liquid prior to oral administration and effervescent tablet. Examples of liquid form adapted to oral administration include, but are not limited to, solutions, suspensions, drinkable solutions, elixirs, sealed phial, potion, drench, syrup and liquor.

According to another embodiment, the compound or the pharmaceutical composition of the invention istopically administered. Topical administration may be particularly advantageous for the treatment of skin cancers. Examples of formulations adapted to topical administration include, but are not limited to, patch, such as, for example, transdermic patch, ointment, gel, cream and the like.

According to another embodiment, for the treatment of conditions of the lungs or of the respiratory tract, aerosol delivery can be used.

Other examples of administration routes include, but are not limited to, nasal, buccal, rectal, vaginal, intratracheal, endoscopic and percutaneous administration.

According to an embodiment, the compound or the pharmaceutical composition of the invention is administered preoperatively, and/or postoperatively.

According to an embodiment, the compound or the pharmaceutical composition of the invention is packaged in unitary dosages. Examples of unitary dosages include, but are not limited to, vials, syringe, pill, caplet, capsule, tablet, sealed phial and pouch containing the compound or the composition of the invention.

Examples of pharmaceutically acceptable vehicles which could be used in the pharmaceutical composition of the invention are well known from the one skilled in the art. The selection of a specific vehicle is dependent on the administration route and on the formulation of the composition of the invention.

Examples of pharmaceutically acceptable vehicles include, but are not limited to water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol™, vegetable oils and the like.

In one embodiment, the subject receiving the compound or the pharmaceutical composition of the invention is an animal, including a human.

According to a first embodiment, the subject receiving the compound or the pharmaceutical composition of the invention is diagnosed with a cancer.

According to a second embodiment, the subject receiving the compound or the pharmaceutical composition of the invention underwent a surgical operation to remove a tumor.

According to a third embodiment, the subject receiving the compound or the pharmaceutical composition of the invention was previously treated with an anti-cancer treatment, or is simultaneously treated with an anti-cancer treatment, or is planned to be treated with an anti-cancer treatment. Examples of anti-cancer treatment include, but are not limited to, chemotherapy and radiotherapy. Indeed, the Inventors herein showed that the compounds of the invention increase the cytotoxic effect of chemotherapeutic agents (FIG. 7).

In the sense of the present invention, these three embodiments are not exclusive: a subject may be concerned by one, two or three of these embodiments.

In one embodiment of the invention, the compound or the pharmaceutical composition of the invention is co-administered to the subject with a chemotherapeutic agent.

Examples of chemotherapeutic agents include, but are not limited to, fluorouracil (5-FU), cyclophosphamide, methotrexate, cisplatin, carboplatin, oxaliplatin, mechlorethamine, chlorambucil, ifosfamide, azathioprine, mercaptopurine, vinca alkaloids (such as, for example, vincristine, vinblastine, vinorelbine or vindesine), taxanes (such as, for example, paclitaxel or docetaxel), podophyllotoxins, topoisomerase inhibitors (such as, for example, irinotecan, topotecan, amsacrine, etoposide, etoposide phosphate or teniposide), actinomycin, anthracyclines (such as, for example, doxorubicin, daunorubicin, valrubicin, idarubicin, epirubicin), bleomycin, plicamycin, mitomycin, abiraterone acetate, abraxane, alemtuzumab, altretamine, amsacrine, asparaginase, azacitidine, bendamustine, bevacizumab, bortezomib, brentuximabvedotin, busulfan, cabazitaxel, capecitabine, carmustine, cetuximab, cladribine, clofarabine, crisantaspase, crizotinib, cytarabine (Ara-C), dacarbazine, dactinomycin, dasatinib, etoposide, fludarabine, gemcitabine, hydroxycarbamide, irinotecan, leucovorin, lomustine, melphalan, mesna, mitotane, mitoxantrone, oxaliplatin, pemetrexed, pentostatin, procarbazine, raltitrexed, rasburicase, satraplatin, streptozocin, tegaflur-uracil, temozolomide, thiotepa, tioguanine, topotecan, trabectedin, treosulfan, and mixtures thereof.

According to an embodiment, the pharmaceutical composition of the invention comprises from about 0.1 mg to about 50 g of the compound of general formula (I) of the invention.

According to an embodiment, the pharmaceutical composition is such that an amount of the compound of general formula (I) of the invention ranging from 1 μg/kg of body weight to 1 g/kg of body weight is administered to the subject.

The present invention also relates to a method for treating a cancer in a subject in need thereof, wherein the method comprises the administration of a pharmaceutical composition comprising a therapeutically effective amount of a compound of general formula (I) to the subject; or the administration of a therapeutically effective amount of a compound of general formula (I) to the subject. In one embodiment of the invention, the method comprises inhibiting the proliferation of cancer cells and/or inducing apoptosis of cancer cells, and/or inhibiting the growth of a tumor and/or inhibiting the number and the development of cancerous cells within the body of the subject and/or inhibiting the occurrence of metastases.

Another object of the invention is a method for inhibiting the proliferation of cancer cells and/or for inducing apoptosis of cancer cells in a subject in need thereof, wherein the method comprises the administration of a pharmaceutical composition comprising a therapeutically effective amount of a compound of general formula (I) to the subject; or the administration of a therapeutically effective amount of a compound of general formula (I) to the subject.

Another object of the invention is a method for inhibiting the growth of a tumor in a subject in need thereof, wherein the method comprises the administration of a pharmaceutical composition comprising a therapeutically effective amount of a compound of general formula (I) to the subject; or the administration of a therapeutically effective amount of a compound of general formula (I) to the subject.

Another object of the invention is a method for inhibiting the number and the development of cancerous cells within the body of the subject in need thereof, wherein the method comprises the administration of a pharmaceutical composition comprising a therapeutically effective amount of a compound of general formula (I) to the subject; or the administration of a therapeutically effective amount of a compound of general formula (I) to the subject.

Another object of the invention is a method for inhibiting the occurrence of metastases in a subject in need thereof, wherein the method comprises the administration of a pharmaceutical composition comprising a therapeutically effective amount of a compound of general formula (I) to the subject; or the administration of a therapeutically effective amount of a compound of general formula (I) to the subject.

According to an embodiment, the method of the invention comprises the injection of the compound or the pharmaceutical composition of the invention, preferably the systemic injection. Examples of systemic injections include, but are not limited to, intravenous, subcutaneous, intramuscular, intradermal and intraperitoneal injection, and perfusion. According to another embodiment, the compound or the composition of the invention is intratumorally or intralesionally injected.

According to another embodiment, the method of the invention comprises the oral administration of the compound or of the pharmaceutical composition of the invention. According to another embodiment, the method of the invention comprises the topical administration of the compound or of the pharmaceutical composition of the invention. According to another embodiment, the method of the invention comprises the administration of the compound or of the pharmaceutical composition of the invention by the use of an aerosol. Other examples of administration routes which could be used in the method of the invention include, but are not limited to, nasal, buccal, rectal, vaginal, intratracheal, endoscopic and percutaneous administration.

According to an embodiment, the method of the invention comprises the preoperative or postoperative administration of the compound or of the pharmaceutical composition of the invention.

In one embodiment of the invention, the method of the invention further comprises administering a therapeutically effective amount of a chemotherapeutic agent. In one embodiment, the chemotherapeutic agent is previously, simultaneously or subsequentially administered.

Examples of chemotherapeutic agents include, but are not limited to, fluorouracil (5-FU), cyclophosphamide, methotrexate, cisplatin, carboplatin, oxaliplatin, mechlorethamine, chlorambucil, ifosfamide, azathioprine, mercaptopurine, vinca alkaloids (such as, for example, vincristine, vinblastine, vinorelbine or vindesine), taxanes (such as, for example, paclitaxel or docetaxel), podophyllotoxins, topoisomerase inhibitors (such as, for example, irinotecan, topotecan, amsacrine, etoposide, etoposide phosphate or teniposide), actinomycin, anthracyclines (such as, for example, doxorubicin, daunorubicin, valrubicin, idarubicin, epirubicin), bleomycin, plicamycin, mitomycin, abiraterone acetate, abraxane, alemtuzumab, altretamine, amsacrine, asparaginase, azacitidine, bendamustine, bevacizumab, bortezomib, brentuximabvedotin, busulfan, cabazitaxel, capecitabine, carmustine, cetuximab, cladribine, clofarabine, crisantaspase, crizotinib, cytarabine (Ara-C), dacarbazine, dactinomycin, dasatinib, etoposide, fludarabine, gemcitabine, hydroxycarbamide, irinotecan, leucovorin, lomustine, melphalan, mesna, mitotane, mitoxantrone, oxaliplatin, pemetrexed, pentostatin, procarbazine, raltitrexed, rasburicase, satraplatin, streptozocin, tegaflur-uracil, temozolomide, thiotepa, tioguanine, topotecan, trabectedin, treosulfan, and mixtures thereof.

In one embodiment, the subject concerned by the method of the invention is an animal, including a human.

According to a first embodiment, the subject concerned by the method of the invention is diagnosed with a cancer. According to a second embodiment, the subject concerned by the method of the invention underwent a surgical operation to remove a tumor. According to a third embodiment, the subject concerned by the method of the invention was previously treated with an anti-cancer treatment, or is simultaneously treated with an anti-cancer treatment, or is planned to be treated with an anti-cancer treatment. Examples of anti-cancer treatment include, but are not limited to, chemotherapy and radiotherapy. In the sense of the present invention, these three embodiments are not exclusive: a subject may be concerned by one, two or three of these embodiments.

The present invention thus also relates to a compound of general formula (I) for treating, or for use in the treatment of, a disease, disorder or condition related to angiogenesis in a subject in need thereof.

Accordingly, the present invention thus also relates to a pharmaceutical composition comprising a therapeutically effective amount of compound of general formula (I) in association with a pharmaceutically acceptable vehicle for treating a disease, disorder or condition related to angiogenesis in a subject in need thereof.

In the present invention, a disease, disorder or condition related to angiogenesis may also be referred as an angiogenic disease, disorder or condition.

The Inventors herein present evidences that the compounds of the invention inhibited the formation of neovessels (FIG. 1D). Surprisingly, they also showed that the compounds of the invention inhibited the adhesion of HUVEC cells (endothelial cells) and that they are cytotoxic for these cells, as they inhibit cell proliferation and induce cell apoptosis (FIGS. 1A, 1B and 1C, FIG. 4, Table 1), thus demonstrating an anti-angiogenic effect.

According to an embodiment, the compound or the pharmaceutical composition of the invention is for use for inhibiting the formation of neovessels. According to another embodiment, the compound or the pharmaceutical composition of the invention is for use for inhibiting the adhesion of endothelial cells. According to another embodiment, the compound or the pharmaceutical composition of the invention is for use for inducing apoptosis of endothelial cells. According to another embodiment, the compound or the pharmaceutical composition of the invention is for use for inhibiting the growth, development or division of endothelial cells. Preferably, the compound or pharmaceutical composition of the invention inhibits endothelial cells proliferation with an IC50 of less than or equal to about 0.5 μM, preferably less than or equal to about 0.2 μM, more preferably less than or equal to about 0.05 μM.

According to an embodiment, the disease, disorder or condition related to angiogenesis is tumor related. Indeed, the growth and metastasis of tumors are directly linked to angiogenesis: the tumor may stimulate the growth of neovessels, which (i) allow the supply of nutrients and oxygen necessary to its growth, and (2) are escape routes for tumors, facilitating the dissemination of metastatic cells through the blood circulation. Therefore, in one embodiment, the disease, disorder or condition related to angiogenesis is a cancerous or non-cancerous tumor or a metastasis of a tumor. Examples of cancers and of cancerous tumors are listed here-above. Examples of non-cancerous tumors include, but are not limited to, angiofibroma and hemangiomas.

According to an embodiment, the disease, disorder or condition related to angiogenesis is an ocular disease, disorder or condition.

In one embodiment, the ocular disease, disorder or condition related to angiogenesis is associated with retinal, peripheral retinal and/or choroidal neovascularization. Examples of such angiogenic diseases include, but are not limited to uveitis, choroiditis, choroidal vasculopathy (including polypoidal choroidal vasculopathy), hypersensitive retinopathy, retinochoroiditis, chorioretinitis, retinal angiomatosis, retinal degeneration, macular degeneration, age related macular degeneration (AMD, including wet and dry AMD), retinal detachment, retinal neovascularisation, proliferative vitreoretinopathy, retinopathy of prematurity (ROP), central serous chorioretinopathy, diabetic retinopathy (including nonproliferative and proliferative diabetic retinopathy), posterior segment trauma, retinal vascular pathologies, retinal telangiectesa, endophthalmitis, macular edema, radiation-induced retinopathy, cystoid macular edema, macular dystrophy (including vitelliform macular dystrophy), diabetic retinopathy, inflammatory pathologies of the retina, ischemic vascular disease, sickle cell anemia, sickle cell retinopathy, sarcoid, syphilis, pseudoxanthomaelasticum, Pagets disease, vein occlusion, artery occlusion, carotid obstructive disease, chronic uveitis/vitritis, fundus flavimaculatus, mycobacterial infections, Lyme's disease, systemic lupus erythematosis, systemic pathologies with implications for the retina, Eales disease, Behcet's disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, toxocariasis, rubella, Vogt-Koyanagi-Harada syndrome, multifocal choroiditis, retinitis pigmentosa, retinischisis, trauma, proliferative diabetic retinopathy, branch retinal vein occlusion, branch retinal arteriolar occlusion, carotid cavernous fistula, sickling hemoglobinopathy (including SS hemoglobinopathy and SC hemoglobinopathy), non-sickling hemoglobinopathy (including AC hemoglobinopathy and AS hemoglobinopathy), IRVAN syndrome (retinal vasculitic disorder characterized by idiopathic retinal vasculitis, an aneurysm, and neuroretinitis), retinal embolization, familial exudative vitreoretinopathy, hyperviscosity syndrome (including leukemia, Waldenstrom macroglobulinemia, multiple myeloma, polycythemia or myeloproliferative disorder), aortic arch syndrome, Eales disease and post-laser complications.

In one embodiment, the ocular disease, disorder or condition related to angiogenesis is associated with corneal neovascularization. Examples of such angiogenic diseases include, but are not limited to diabetic retinopathy, retinopathy of prematurity, corneal graft rejection, neovascular glaucoma and retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, trauma, rheumatoid arthritis, systemic lupus, polyarteritis, Wegeners sarcoidosis, Scleritis, Steven's Johnson disease, periphigoid radial keratotomy, and corneal graph rejection.

In one embodiment, the ocular disease, disorder or condition related to angiogenesis is selected from the group comprising diseases associated with rubeosis (neovascularization of the angle) and diseases caused by the abnormal proliferation of fibrovascular or fibrous tissue including all forms of proliferative vitreoretinopathy, whether or not associated with diabetes.

According to an embodiment, the disease, disorder or condition related to angiogenesis is a disease of the renal and/or urogenital tract. Examples of angiogenic diseases of the renal and/or urogenital tract include, but are not limited to, diabetic nephropathy, endometriosis, haemolytic uremic syndrome, hypertensive nephrosclerosis, inflammatory renal disease, such as glomerulonephritis (including mesangioproliferative glomerulonephritis), inhibition of ovulation and corpus luteum formation, inhibition of implantation and inhibition of embryo development in the uterus and nephrotic syndrome.

According to an embodiment, the disease, disorder or condition related to angiogenesis is a disease of the digestive tract. Examples of angiogenic diseases of the digestive tract include, but are not limited to, inflammatory bowel disease.

According to an embodiment, the disease, disorder or condition related to angiogenesis is a disease of the respiratory tract. Examples of angiogenic diseases of the respiratory tract include, but are not limited to, sarcoid, sarcoidosis, Wegeners sarcoidosis, lung inflammation and chronic asthma.

According to an embodiment, the disease, disorder or condition related to angiogenesis is a skin disease. Examples of angiogenic skin diseases include, but are not limited to, psoriasis, acne rosacea, Kaposi's sarcoma, leukemia, Osier-Weber syndrome (Osler-Weber-Rendu), pemphigoid, pseudoxanthomaelasticum, pyogenic granuloma, scleroderma, Steven's Johnson disease and syphilis.

According to an embodiment, the disease, disorder or condition related to angiogenesis is an autoimmune disease. Examples of autoimmune diseases related to angiogenesis include, but are not limited to, chronic asthma, Crohn's disease, pemphigoid, scleroderma, Sogrens syndrome and systemic lupus.

According to an embodiment, the disease, disorder or condition related to angiogenesis is consecutive to a graft. Examples of reactions related to angiogenesis following the graft of an organ or tissue include, but are not limited to, immune rejection of transplanted organs or tissues (including corneal graft rejection), corneal graft neovascularization and diseases occurring after transplants.

According to an embodiment, the disease, disorder or condition related to angiogenesis is a post-surgery reaction, a hyperproliferation or a hypertrophy. Examples of such diseases include, but are not limited to, abnormal proliferation of fibrovascular tissue, hypertrophic scars and hypertrophy following surgery.

According to an embodiment, the disease, disorder or condition related to angiogenesis is a disease of bones or joints. Examples of diseases of bones or joints related to angiogenesis include, but are not limited to, Pagets disease, osteoarthritis, hemophilic joints, psoriatic arthritis, rheumatoid arthritis and nonunion fractures.

According to an embodiment, the disease, disorder or condition related to angiogenesis is abnormal wound healing.

According to an embodiment, the disease, disorder or condition related to angiogenesis is chronic inflammation.

According to an embodiment, the disease, disorder or condition related to angiogenesis is a disease of the cardiovascular system or of blood. Examples of diseases of the cardiovascular system or of blood related to angiogenesis include, but are not limited to, arterial arteriosclerosis, arteriovenous malformations, artery occlusion, atherosclerosis, carotid obstructive disease, diseases and disorders characterized by undesirable vascular permeability, e.g., edema associated with brain tumors, ascites associated with malignancies, hyperviscosity syndromes, pericardial effusion, such as that associated with pericarditis, and pleural effusion, polyarteritis, preeclampsia, vascular adhesions and vein occlusion.

According to an embodiment, the disease, disorder or condition related to angiogenesis is an infection. Examples of infections causing angiogenic disorders include, but are not limited to, bacterial infection, bacterial ulcers, fungal infection, fungal ulcers, Herpes simplex infections, Herpes zoster infections, Lyme's disease, Mycobacteria infections other than leprosy, parasitic diseases, protozoan infections, toxoplasmosis and viral infection.

According to an embodiment, the compound or the pharmaceutical composition of the invention may be administered by injection, oral administration, topical administration, implant, aerosol, or by nasal, buccal, vaginal, rectal, intratracheal, endoscopic or percutaneous administration.

According to an embodiment, the compound or the pharmaceutical composition of the invention is injected. Examples of formulations adapted to injections include, but are not limited to, liquid solutions or suspensions, solid forms suitable for solution in, or suspension in, liquid prior to injection. Examples of injections include, but are not limited to, systemic, intravenous, intraocular, subcutaneous, intramuscular, intradermal, intraperitoneal, intratumoral and intralesional injection, and perfusion. According to an embodiment, when injected, the compound or the pharmaceutical composition of the invention is sterile. Methods for obtaining a sterile pharmaceutical composition include, but are not limited to, GMP synthesis (GMP stands for “Good manufacturing practice”).

According to another embodiment, the compound or the pharmaceutical composition of the invention is orally administered. Examples of formulations adapted to oral administration include, but are not limited to, solid forms, liquid forms and gels. Examples of solid forms adapted to oral administration include, but are not limited to, pill, tablet, capsule, soft gelatin capsule, hard gelatin capsule, caplet, compressed tablet, cachet, wafer, sugar-coated pill, sugar coated tablet, orodispersing/orodisintegrating tablet, powder, solid forms suitable for solution in, or suspension in, liquid prior to oral administration and effervescent tablet. Examples of liquid form adapted to oral administration include, but are not limited to, solutions, suspensions, drinkable solutions, elixirs, sealed phial, potion, drench, syrup and liquor.

In one embodiment of the invention, the disease, disorder or condition is an ocular disease. According to this embodiment, the compound or the pharmaceutical composition of the invention may be injected, for example intraocularly or periocularly injected. By “intraocularly injected” is meant, in the present invention, injected directly in the interior of the eye, wherein the interior of the eye means any area located within the eyeball, and which generally includes, but is not limited to, any functional (e.g. for vision) or structural tissues found within the eyeball, or tissues or cellular layers that partially or completely line the interior of the eyeball. Specific examples of such areas include the anterior chamber, the posterior chamber, the vitreous cavity, the choroid, the macula, and the retina, and blood vessels and nerves which vascularize or innervate a posterior ocular region or site. By “periocularly injected” is meant, in the present invention, injected in the tissues adjacent to the eyeball, such as, for example, peribulbar, laterobulbar, subconjonctival, sub-tenonorretrobulbar injection. Examples of formulations adapted to intraocular or periocular injection include, but are not limited to, solutions, emulsions or suspensions. According to this embodiment, the compound or the pharmaceutical composition of the invention may also be topically applied. Examples of formulations adapted to topical administration to the eye include, but are not limited to, ointments and eye drops. According to this embodiment, the compound or the pharmaceutical composition of the invention may also be an intraocular implant. Examples of intraocular implant include, but are not limited to, intraocular lenses.

According to another embodiment, the compound or the pharmaceutical composition of the invention is topically administered. Topical administration may be particularly advantageous for the treatment of skin conditions. Examples of formulations adapted to topical administration include, but are not limited to, patch, such as, for example, transdermic patch, ointments, eye drops, gels, cream and the like.

According to another embodiment, the compound or the pharmaceutical composition of the invention is administered by an aerosol. Treatments using an aerosol are of particular interest for the treatment of conditions of the lungs or of the respiratory tract.

According to an embodiment, the compound or the pharmaceutical composition of the invention is a suppository. The use of suppositories is of particular interest for the treatment of diseases of the urogenital tract and/or of the digestive tract.

According to an embodiment, the compound or the pharmaceutical composition of the invention is packaged in unitary dosages. Examples of unitary dosages include, but are not limited to, vials, syringe, pill, caplet, capsule, tablet, sealed phial, suppositories, and pouch containing the composition of the invention.

According to an embodiment, the compound or the pharmaceutical composition of the invention is administered preoperatively, and/or postoperatively.

Examples of pharmaceutically acceptable vehicles which could be used in the pharmaceutical composition of the invention are well known from the one skilled in the art. The selection of a specific vehicle is dependent on the administration route and on the formulation of the composition of the invention.

Examples of pharmaceutically acceptable vehicles include, but are not limited to water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol™, vegetable oils and the like.

In one embodiment, the subject receiving the compound or the pharmaceutical composition of the invention is an animal, including a human.

According to an embodiment, the subject receiving the compound or the pharmaceutical composition of the invention is diagnosed with a disease, disorder or condition related to angiogenesis.

According to a second embodiment, the subject receiving the compound or the pharmaceutical composition of the invention was previously treated with another anti-angiogenic treatment, or is simultaneously treated with another anti-angiogenic treatment, or is planned to be treated with another anti-angiogenic treatment.

In the sense of the present invention, these two embodiments are not exclusive: a subject may be concerned by only one or two of these embodiments.

According to an embodiment, the pharmaceutical composition of the invention comprises from about 0.1 mg to about 50 g of the compound of formula (I) of the invention.

According to an embodiment, the pharmaceutical composition is such that an amount of the compound of formula (I) of the invention ranging from 1 μg/kg of body weight to 1 g/kg of body weight is administered to the subject.

The present invention also relates to a method for treating an angiogenic disease, disorder or condition in a subject in need thereof, wherein the method comprises the administration of a therapeutically effective amount of a compound of general formula (I) or of a pharmaceutical composition comprising a therapeutically effective amount of a compound of general formula (I) to the subject. In one embodiment of the invention, the method comprises inhibiting the formation of neovessels and/or inhibiting the adhesion of endothelial cells and/or inhibiting the growth, development or division of endothelial cells, and/or inducing apoptosis of endothelial cells in the subject.

Another object of this invention is a method for inhibiting the formation of neovesselsin a subject in need thereof, wherein the method comprises the administration of a therapeutically effective amount of a compound of general formula (I) or of a pharmaceutical composition comprising a therapeutically effective amount of a compound of general formula (I) to the subject.

Another object of this invention is a method for inhibiting the adhesion of endothelial cells in a subject in need thereof, wherein the method comprises the administration of a therapeutically effective amount of a compound of general formula (I) or of a pharmaceutical composition comprising a therapeutically effective amount of a compound of general formula (I) to the subject.

Another object of this invention is a method for inhibiting the growth, development or division of endothelial cells, and/or for inducing apoptosis of endothelial cells in a subject in need thereof, wherein the method comprises the administration of a therapeutically effective amount of a compound of general formula (I) or of a pharmaceutical composition comprising a therapeutically effective amount of a compound of general formula (I) to the subject.

According to an embodiment, the method of the invention comprises the injection, the oral, topical, nasal, buccal, vaginal, rectal, intratracheal, endoscopic or percutaneous administration, or the administration by the use of an implant or of an aerosol of the compound or of the pharmaceutical composition of the invention.

In one embodiment, the subject concerned by the method of the invention is an animal, including a human.

According to an embodiment, the subject concerned by the method of the invention is diagnosed with a disease, disorder or condition related to angiogenesis. According to a second embodiment, the subject concerned by the method of the invention was previously treated with another anti-angiogenic treatment, or is simultaneously treated with another anti-angiogenic treatment, or is planned to be treated with another anti-angiogenic treatment. In the sense of the present invention, these two embodiments are not exclusive: a subject may be concerned by only one or two of these embodiments.

According to an embodiment, the method of the invention comprises the preoperative and/or the postoperative administration of the compound or of the pharmaceutical composition of the invention.

### BRIEF DESCRIPTION OF THE DRAWINGS

FIG. 1 is a combination of graphs and pictures showing the in vitro effect of the compounds 1, 2 and 3 of the invention on cell adhesion, cell survival and vessel formation. (A) Graphs showing the effect of the compound 1 of the invention on the adhesion and on the survival of HUVEC and MDAMB-231 cells. (B) Graphs showing the effect of the compound 2 of the invention on the adhesion and on the survival of HUVEC and MDAMB-231 cells. (C) Graphs showing the effect of the compound 3 of the invention on the adhesion and on the survival of HUVEC and MDAMB-231 cells. (D) Images showing the effect of the compounds 1 and 2 of the invention on the formation of vessels.

FIG. 2 is a combination of graphs showing the effects of compounds 1 and 2 of the invention on endothelial cells proliferation and apoptosis. (A) Compounds 1 and 2 inhibited the cell proliferation of endothelial cells in a time-dependent manner. Cell proliferation was evaluated by WST-1 assay. (B) Compounds 1 and 2 induced the apoptosis of cells after 72 h of treatment. Apoptotic cells represented Annexin V-positive (early stage of apoptosis) Annexin V-PI double-positive (late stage of apoptosis) cells as determined by flow cytometry. Histograms represent the mean±SD of at least three independent experiments.

FIG. 3 is a combination of graphs showing the effects of compounds 1 and 2 of the invention on endothelial cells migration and on tube formation and maintenance. (A) Effect of compounds of the invention on endothelial cells migration. Difference of migration speed, based on cell motility tracks analysis of control and treated HUVEC using Metamorph 7 software. Histogram represents the mean±SD of at least three independent experiments. (B) and (C) Effect of compounds of the invention on the formation of capillary-like structures. (B) Compounds inhibited the tube formation. HUVEC cells were seeded on collagen with VEGF-A₁₆₅-stimulation in presence or absence of compounds 1 and 2. Tube-like structures were visualized 24 h later and were quantified by number of branching points. (B) Tube maintenance in VEGF-A₁₆₅-treated HUVEC cells cultured in collagen is inhibited by compounds 1 and 2, after 24 h of treatment, in contrast to the control.

FIG. 4 is graphs showing the inhibitory effect of the compounds 1 and 2 of the invention on HeLa cells following cytotoxicity.

FIG. 5 is a combination of graphs showing the effects of the compounds 1 and 2 of the invention on breast cancer cells proliferation and apoptosis. (A) Compounds 1 and 2 inhibited the cell proliferation of breast cancer cells in time-dependent manner. Cell proliferation was evaluated by WST-1 assay. (B) Compounds 1 and 2 induced the apoptosis of cells after 72 h treatment. Apoptotic cells represented Annexin V-positive (early stage of apoptosis) Annexin V-PI double-positive (late stage of apoptosis) cells as determined by flow cytometry. Histograms represent the mean±SD of at least three independent experiments.

FIG. 6 is a graph showing the effect of the compounds 1 and 2 of the invention on breast cancer cells migration. The graph shows the difference of migration speed based on the quantification of the motility levels of individual cell by establishing the trajectory of each cell using Metamorph 7 software. Histogram represents the mean±SD of at least three independent experiments.

FIG. 7 is a combination of graphs showing the effect of the compounds 1 and 2 of the invention on breast cancer cells proliferation in combination with chemotherapeutic agent. Breast cancer cells were incubated in medium containing 5-Fluorouracil in absence or presence of compounds 1 (A) and 2 (B). Cell proliferation was determined after 72 h by WST-1 assay. The IC₅₀ values were determined with a curve-fitting analysis (nonlinear regression curve). Data are expressed as mean±SD of at least three experiments; each performed using triplicate determinations at each concentration of the compound.

FIG. 8 is a combination of graphs showing the in vivo efficiency of the compound 1 of the invention. (A) Effect of the compound ion body weight. (B) Effect of the compound ion tumor growth. (C) Kaplan-Meier survival curves showing the effect of the compound ion survival of mouse.

### EXAMPLES

### Example 1

### Synthesis of the Compounds of the Invention

### Synthesis of N-[5-(1H-benzimidazol-2-yl)-2-methylphenyl]-N′-(2,3-dihydro-1,4-benzodioxin-6-ylcarbonyl)thiourea (Compound 1 of the Invention)

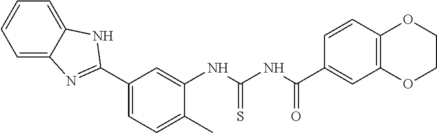 [SMILES: Cc1ccc(-c2nc3ccccc3[nH]2)cc1NC(=S)NC(=O)c1ccc2c(c1)OCCO2]

A solution of 4-Methyl-3-nitrobenzaldehyde (3.34 g, 20.2 mmol, 1 eq) and sodium bisulfate (11.51 g, 60.6 mmol, 3 eq) in 10 ml of dimethylformamide (DMF), was refluxed for 1 hr, then a solution of o-phenylenediamine (2.18 g, 20.2 mmol, 1 eq) in 10 ml of DMF was added drop wise and the mixture was refluxed for a further 3 h. The solution was cooled to room temperature and poured into ice-water. The organic layer was then washed with brine and dried over Na₂SO₄ then evaporated to dryness. The residue obtained was triturated in order to eliminate impurities and the solid was collected by filtration to give 5(1H-benzoimidazol-2-yl)-2-methyl-phenylamine as a brown powder (3.04 g, yield 67%). NMR (DMSO-d6): 2.14 (s, 3H, CH₃); 5.07 (s, 2H, NH₂); 7.1-7.6 (m, 7H, H—Ar); 12.65 (s, 1H, NH).

1,4-Benzodioxane-6-carboxylic acid (4.5 g, 25 mmol) was refluxed in thionyl chloride (25 ml) for 3 hr, the solution was then evaporated to dryness to give 1,4-benzodioxin-6-carbonyl chloride as a pale solid (4.9 g, yield: 100%).

A solution of benzodioxin-6-carbonyl chloride (2.2 g, 10 mmol, 1 eq) and ammonium thiocyanate (0.84 g, 10 mmol, 1 eq) in dry 8 ml acetone was refluxed for 1 hr. The mixture was then cooled to room temperature and a solution of 5(1H-benzoimidazol-2-yl)-2-methyl-phenylamine (2.34 g, 10 mmol, 1 eq) in 18 ml dry acetone was added. The mixture was refluxed for another 1 hr, then the solution was cooled and 100 ml of water was added. The precipitate was collected by filtration and the crude product was recrystallized in EtOH to obtained 1 white powder (0.870 g, yield 63%). NMR (DMSO-d6): 2.34 (s, 3H, CH₃); 4.34 (s, 4H, 2*CH₂O); 7.0-8.1 (m, 10H, H—Ar); 8.34 (s, 1H, NH); 11.54 (s, 1H, NH); 12.40 (s, 1H, NH). MS (ESI+): 445 (M+H⁺) found for 444 calculated. HPLC (Vydac C18, 4.6*250 mm. A: H₂O with 0.1% TFA, B: 70% acetonitrile with 0.09% TFA. Flow 1 ml/min): Rt: 14.9 min.

### Synthesis of N-[3-(1H-benzimidazol-2-yl)phenyl]-N′-(2,3-dihydro-1,4-benzodioxin-6-ylcarbonyl)thiourea (Compound 2 of the Invention)

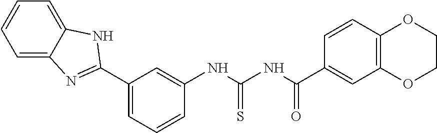 [SMILES: O=C(NC(=S)Nc1cccc(-c2nc3ccccc3[nH]2)c1)c1ccc2c(c1)OCCO2]

A solution of 3-nitrobenzaldehyde (2.90 g, 26.5 mmol, 1 eq) and sodium bisulfate (15.10 g, 79.5 mmol, 3 eq) in 10 ml of DMF, was refluxed for 1 h, then a solution of o-phenylenediamine (4.00 g, 26.5 mmol, 1 eq) in 10 ml of DMF was added dropwise and the mixture was refluxed for a further 3 h. The solution was cooled to room temperature and poured into ice-water. The organic layer was then washed with brine and dried over Na₂SO₄ then evaporated to dryness. The residue obtained was triturated in order to eliminate impurities and the solid was collected by filtration to give 5(1H-benzoimidazol-2-yl)-2-methyl-phenylamine as a brown powder (2.00 g, yield 39%). NMR (DMSO-d6): 6.07 (s, 2H, NH₂); 7.2-7.6 (m, 8H, H—Ar); 12.6 (s, 1H, NH).

A solution of benzodioxin-6-carbonyl chloride (0.97 g, 4.4 mmol, 1 eq) and ammonium thiocyanate (0.37 g, 4.4 mmol, 1 eq) in dry 6 ml acetone was refluxed for 1 hr. The mixture was then cooled to room temperature and a solution of 5(1H-benzoimidazol-2-yl)-2-methyl-phenylamine (0.96 g, 4.4 mmol, 1 eq) in 15 ml dry acetone was added. The mixture was refluxed for another 1 hr, then the solution was cooled and 100 ml of water was added. The precipitate was collected by filtration and the crude product was recrystallized in EtOH to obtained 2 white powders (0.465 g, yield 27%). NMR (DMSO-d6): 4.33-4.37 (m, 4H, 2*CH₂O); 7.00-8.12 (m, 11H, H—Ar); 8.41 (s, 1H, NH); 11.58 (s, 1H, NH); 12.86 (s, 1H, NH). MS (ESI+): 431 (M+H⁺) found for 430 calculated. HPLC (Vydac C18, 4.6*250 mm. A: H₂O with 0.1% TFA, B: 70% acetonitrile with 0.09% TFA. Flow 1 ml/min): Rt: 13.8 min.

### Example 2

### Characterization of the Compounds 1, 2 and 3 of the Invention

| Product | LogP value | MS | Melting point |
|---|---|---|---|
| 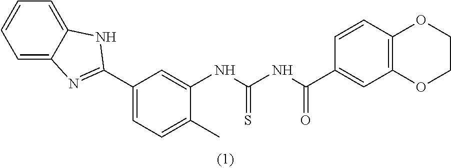 [SMILES: Cc1ccc(-c2nc3ccccc3[nH]2)cc1NC(=S)NC(=O)c1ccc2c(c1)OCCO2] | 3.15 | MH + 445 | 202° C. |
| 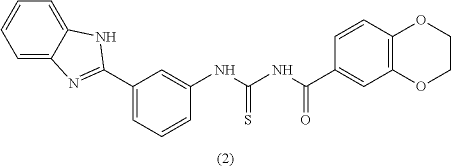 [SMILES: O=C(NC(=S)Nc1cccc(-c2nc3ccccc3[nH]2)c1)c1ccc2c(c1)OCCO2] | 2.94 | MH + 431 | 212° C. |
| 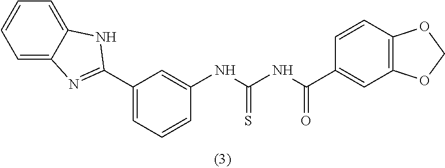 [SMILES: O=C(NC(=S)Nc1cccc(-c2nc3ccccc3[nH]2)c1)c1ccc2c(c1)OCO2] | 3.061 | MH + 417 | 228° C. |

LogP values were calculated using jlogP program, available at the web address:http://www.v1s3d.com/programs.html#section9.

### Example 3

### Biological Examples

### Methods

### Cell Culture

HUVEC (Lonza, Switzerland, cat no C2517A) were maintained in EBM®-2 (Lonza, France, cat no: CC-3162) supplemented with a kit including 2% foetal bovine serum, human fibroblast growth factor-2 (hFGFB), human epidermal growth factor (hEGF), insulin like growth factor-1 (R3-IGF-1), hydrocortisone, gentamicin and amphotericin-B, heparin and ascorbic acid (Lonza, France, cat no: CC-4176).

Several cancer cell lines were used: breast cancer cells (MDA-MB-231 (ATTCC® Number HTB-26™), MDA-MB-436 (ATTCC® Number HTB-130™), MDA-MB-438, BT-474 (ATTCC® Number HTB-20™)); cervical cancer cells (HeLa (ATTCC® Number CCL-2™)); colon cancer cells (HCT-116 (ATTCC® Number CCL-247™) and HT-29 (ATTCC® Number HTB-38™)); osteosarcoma cells (U2OS (ATTCC® Number HTB-96™)); pancreas cancer cells (MIA PaCa-2 (ATTCC® Number CRL-1420™) and Panc-10.5 (ATTCC® Number CRL-2547™)); prostate cancer cells (PC-3 (ATTCC® Number CRL-1435™) and LNCaP (ATTCC® Number CRL-1740™)); brain cancer cells (U87 glioblastoma (ATTCC® Number HTB-14™) and SH-SYSY neuroblastoma (ATTCC® Number CRL-2266™)).

All the cancer cell lines were maintained in cultured in DMEM GlutaMAX™ (GIBCO Invitrogen, France, cat no 31966047) enhanced with 10% foetal bovine serum (GIBCO Invitrogen, France, cat no 10270-106) and 1% penicillin G/streptomycin (GIBCO Invitrogen, France, cat no 15140-122). Only Panc-10.5 cells were cultivated in RPMI 1640 medium (Gibco) supplemented with 2 mM L-glutamine, 1.5 g/L sodium bicarbonate, 4.5 g/L glucose, 10 mM HEPES, 1 mM sodium pyruvate, 10 Units/ml human insulin, 85% fetal bovine serum (SVF): 15% (75 ml in 500 ml of medium).

Cells were grown as subconfluent monolayer and cultured in 75 cm² flasks at 37° C., 5% CO₂ in a humidified atmosphere, and medium was changed every two days. Only cells from passages 2-6 were used for the experiments.

After observation of cells under the microscope, old media were removed and the adherent cells washed gently twice with PBS (Phosphate Buffer Saline, —Ca²⁺, —Mg²⁺, Lonza cat no: 17-512F/12). All cells were detached with 3 ml of trypsin/EDTA (chelator of calcium and magnesium ions) (Lonza), incubated for 5 min at 37° C., and examined using a microscope to ensure that the cells were detached and floating. Then, 3 ml TNS (Trypsin Neutralizing Solution, Lonza, France, cat no CC-5002) or of culture medium were added to inactivate the trypsin, and the cell suspension was transferred in a falcon and centrifuged for 7 min at 1400 rpm. The supernatant was removed and the cell pellet resuspended in 10 μl of culture medium. 50 μl of this solution were transferred in 20 μl of isotonic solution and the cells were counted by count cellular (Beckman Coulter). At day 0, 200 μl of cellular suspension (3.10³ cells/well for the HUVEC cells, breast cancer cells (MDA-MB-231 1.10⁴ cells/well, MDA-MB-436 1.10⁴ cells/well, MDA-MB-438 2.10⁴ cells/well, BT-474 2.5.10⁴ cells/well); cervical cancer cells (HeLa 5.000 cells/well); colon cancer cells (HCT-116 5.10³ cells/well, HCT-29 1.1.10⁴ cells/well); osteosarcoma cells (U2OS 8.10³ cells/well); pancreas cancer cells (MIA PaCa-2 5.10³ cells/well, Panc-10.5 1.5.10⁴ cells/well); prostate cancer cells (PC-3 4.10³ cells/well, LNCaP 2.10⁴ cells/well); brain cancer cells (U87 5.10³ cells/well, SH-SYSY 8.10⁴ cells/well)) were then plated on 96 well plates and incubated at 37° C. in a humidified atmosphere of 5% CO₂ for 24 h.

At day 1, the compounds 1 or 2 of the invention were added at increasing concentrations (0.1-10 μM) in the wells and plates were incubated at 37° C. in a humidified atmosphere of 5% CO₂ for 72 h.

### Adhesion Test

The crystal violet (hexamethyl pararosaniline chloride) is used as biologic colorant. It enters in the living cells, allowing the visualisation of the dark purple adherent living cells on the bottom of the plate.

After an incubation period of 72 h, the adherent cells were fixed with 50 ml paraformaldehyde

3% for 15 min at room temperature. The medium and non-adherent cells were removed, and the cells were washed extensively with 100 ml PBS (+Ca²⁺ and +Mg²⁺) (Lonza) and then stained with 50 μl of crystal violet 0.04% for 30 min at room temperature. The cells were washed gently 3 times with 100 μl PBS and were permeabilised with 100 μl triton 2% for 15 min. The microplate was stirred to obtain a homogenous solution and absorbance was quantified at 580 nm by Microplate Manager 5.2.

The percentage of inhibition is calculated as follow: % inhibition=100−[(DOₛₐₘₚₗₑ/DO_{control})*100].

### Proliferation Assay

The cell proliferation WST-1 assay is a colorimetric assay for the quantification of cell proliferation and viability, based on the enzymatic cleavage of the tetrazolium salt WST-1 added to the culture medium, to a water soluble colored formazan dye, detected by absorbance at 450 nm, by mitochondrial succinate-tetrazolium reductase in viable cells. An augmentation in enzyme activity will then lead to an increase in the amount of formazan dye formed, which directly correlates to the number of metabolically active cells in the culture.

After an incubation period of 72 h, at day 4, were prepared the 19 ml medium with reagent WST-1 1 ml (10 ml WST-1 in 200 ml well). The medium was removed from the 96 microplate and 200 μl of solution WST-1/medium were added for each well. The cells were incubated at 37° C. in a humidified atmosphere of 5% CO₂ for 1 h 30 and the absorbance at 450-655 nm was measured by Microplate Manager 5.2 (Bio-Rad).

### Apoptosis Assay

The level of apoptosis in endothelial (HUVEC) and breast cancer cells (MDA-MB-231) was detected by Annexin V-FITC Apoptosis Detection Kit (ref 556547, BD Biosciences, France) according to the manufacturer's instructions. Briefly, HUVEC and MDA-MB-231 cells were treated separately with DMSO (control) and the compounds of the invention at the concentration corresponding to the IC₅₀, obtained by the proliferation assay, for 72 h. After treatment, all cell including both detached and attached cells were collected, centrifugated at 14000 rpm for 5 min and the apoptotic cells were detected by staining with Annexin V-FITC and (propidium iodide) PI. The cells were resuspended in 100 μl Annexin V binding buffer (10 mM Hepes/NaOH, pH 7.4, 140 mM NaCl, 25 mM CaCl₂) containing 5 μl of Annexin V-FITC and 5 μl of PI (propidium iodide). Samples were gently mixed and incubated for 2 h on ice and in the dark. Viable and apoptotic cell were detected by flow citometry (FACS Calibur Becton Dickinson, France). Apoptotic cells include both the early apoptotic portion (Annexin V-positive) and the late apoptotic portion (Annexin V- and PI-positive). Staurosporine is used as a positive control of apoptosis induction.

### Motility Assay (Videomicroscopy)

Endothelial (HUVEC) and breast cancer cells (MDA-MB-231) were seeded in 35 mm diameter tissue culture dishes at 3×10⁴ and 6×10⁴ respectively and treated separately with DMSO (control) and compounds of the invention (treatment) for 24 h, at the concentration of IC₅₀ obtained with the proliferation assay.

The dishes were placed in an enclosed warming incubator on the motorized stage of a computer-controlled Eclipse Ti inverted microscope (Nikon Instruments Europe) equipped with a CCD Cool-SNAP HQ2 camera (Roper Scientific, USA) and a PC workstation. The incubator allowed samples to be kept at 37° C. in a humidified atmosphere containing 5% CO₂, 95% air. Phase images were recorded at 100× every 5 min simultaneously over several fields from 0 till 24 h (288 frames). Cell motility was tracked and analyzed for at least 25 cells in each field over three independent experiments. The total distance of migration and the speed of migration were analyzed. The degree of persistence of movement was defined as the ratio between the linear distance and the total distance covered by the cells. Image acquisition and analyses were carried out using Metamorph 7 software (Universal Imaging).

### Inhibition of VEGF on NRP-1, NRP-2 or VEGF-R1 Interaction

Elisa test was performed covering a 96-well microplate (BD Biosciences, France, cat no 353046) surface with 100 μl of 1×PBS, pH 7.4 with Ca²⁺ and Mg²⁺, (Lonza, France, cat no 17-513F/12) solution containing 0.2 μg/ml of VEGF-R1 (R&D Systems, UK, cat no 321-FL-050/CF) or 2 μg/ml of NRP-1 (R&D Systems, UK, cat no 3870-N1-025) or NRP-2 (R&D Systems, UK, cat no 321-FL). The plate was sealed and incubated overnight at 4° C. After 3 washes with 200 μl of 1×PBS 0.5% (v/v) Tween 20 (Sigma, France, cat no 34078) (Buffer A), the plate was blocked by adding 200 μl of 1×PBS with 0.5% (w/v) BSA (Eurobio, France, cat no GAUBSA01-65) and agitated at 37° C. for 2 h. After washing five times with Buffer A, 50 μl/well of the compounds of the invention at 10 μM were added for the specific signal and 50 μl/well for the non-specific and max binding signal and incubated at 37° C. for 1 hour, shacking. 50 μl of btVEGF (R&D Systems, UK, cat no NFVEO) at 200 μg/ml for NRP-1/4 μg/ml Heparin (Sigma, USA, cat no H3393-10KU), 300 μg/ml for NRP-2 and 100 μg/ml for VEGF-R1 solution was added to each well; the plate was sealed and incubated at 37° C. for 2 h, shacking. The last incubation was carried out, after five washes with Buffer A, adding 100 μl/well Streptavidin-Horseradish Peroxidase (Amersham, Switzerland, cat no RPN4401) diluted 1:8000 with buffer A. After 1 h shacking at 37° C. under obscurity, the plate was washed 5 times with buffer A and 100 μl of SuperSignal west picochemiluminescent substrate (Interchim, USA cat no 34078) were added. Luminescence was quantified with an EnVision™ 2101 Multilabel reader (Perkin Elmer, USA) and the data analyzed by Wallac Envision manager software. Signal is given as relative light units (RLU). Non-specific binding is defined as the signal measured in the absence of receptor coated on the microplate. The specific binding was calculated as the difference between total binding and non-specific binding. The percentage of inhibition was calculated as following:

$$Inhibition \% = 100 \times \frac{( Signal measured - non specific binding signal )}{\begin{matrix}( Max binding signal obtained without inhi bitor - \\ non specific binding signal without receptor )\end{matrix}}$$

### Tube Formation Assay

Collagen type I (1 mg/ml, BD) was mixed with 0.1N NaOH, 0.1% bicarbonate and 10× in medium EBM-2 and kept on ice until placed into the wells. The assay was prepared in 96-well plate format and the bottom layer of collagen was formed by adding 70 μL of the solution per well and allowing subsequent gelation at 37° C. for 30 min. The HUVEC cells were then seeded on collagen at 8.10⁵ cells per well. After monolayer formation (one day), a second layer of collagen 200 μL per well was prepared according to the same protocol and gently placed on top of the cell layer. The compounds of the invention were added to the cells at the adhesion step (concentration corresponding to the Inhibitory Concentration 50 (IC₅₀)). After 24 and 48 h of incubation at 37° C., tube formation was observed and pictures were taken.

### Tube Maintenance Assay

A 96-well plate was coated with 70 μl of collagen/media solution (NaOH 0.1N, 48 μL 10× Na₂HCO₃ and 0.5 mL EBM2) and allowed to solidify at 37° C. for 3 min. HUVEC were seeded at a density of 8×10⁵ cells for well in 100 μl complete culture medium. After confluent monolayer formation, a second collagen gel layer with EBM2 supplemented with 50 ng/ml of VEGF-A₁₆₅ was added over cells and plates were incubated for 24 h at 37° C. and 5% CO₂ to allow tube formation. Then, HUVEC tubes were treated with the compounds of the invention at the concentration of IC₅₀ for additional 24 h before observation.

### In Vivo Studies

Female (NOD-SCID) mice 5-6 weeks old were injected subcutaneously with MDA-MB-231 cells (5.10⁶) at a single site on their right flanks. Tumors were allowed to grow (19 days) to less than 110 mm before being pair-matched by tumor size into treatment groups (8 mice/group). One group was treated with vehicles (8 mice/group receiving cremofor 12.5%/ethanol 12.5%/H2O), one group was treated with 10 mg/kg of the compound 1 of the invention (8 mice/group receiving compound 1+cremofor 12.5%/ethanol 12.5%/H₂O), one group was treated with 50 mg/kg of the compound 1 of the invention (9 mice/group receiving compound 1+cremofor 12.5%/ethanol 12.5%/H₂O). All groups received daily by oral gavages for 5 consecutive days/weeks approximately 100 μL of a solution containing the compound 1 of the invention. Mice were weighed regularly to assess toxicity of the treatments and the tumors were measured with calipers (width×width×length×Pi/6) to determine tumor growth. Mice survival was assessed during the study and metastatic development was investigated in lung, spleen and liver.

### Statistical Analysis

Data reported here are the means±SD of three/five independent experiments, each performed in triplicate. The statistical significance of results was evaluated by ANOVA test, with probability values *p=0.05, **p=0.01, ***p=0.001, being considered as significant.

### Results

The presented results showed that the compounds 1, 2 and 3 of the invention were cytotoxic for endothelial cell lines (HUVEC cells) in culture.

IC₅₀ values of the inhibition of HUVEC cells proliferation are shown in the Table 1 below:

TABLE 1

| Compound of | Anti-proliferativeactivity on |
|---|---|
| the invention | HUVEC cells (IC₅₀, μM) |
| Compound 1 | 0.2 ± 0.01 |
| Compound 2 | 0.05 ± 0.01 |

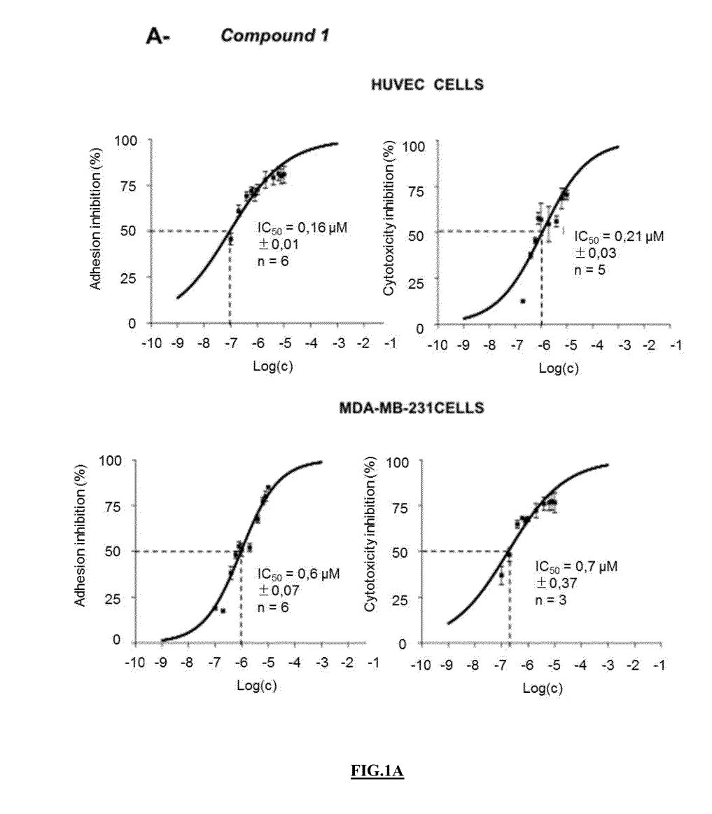

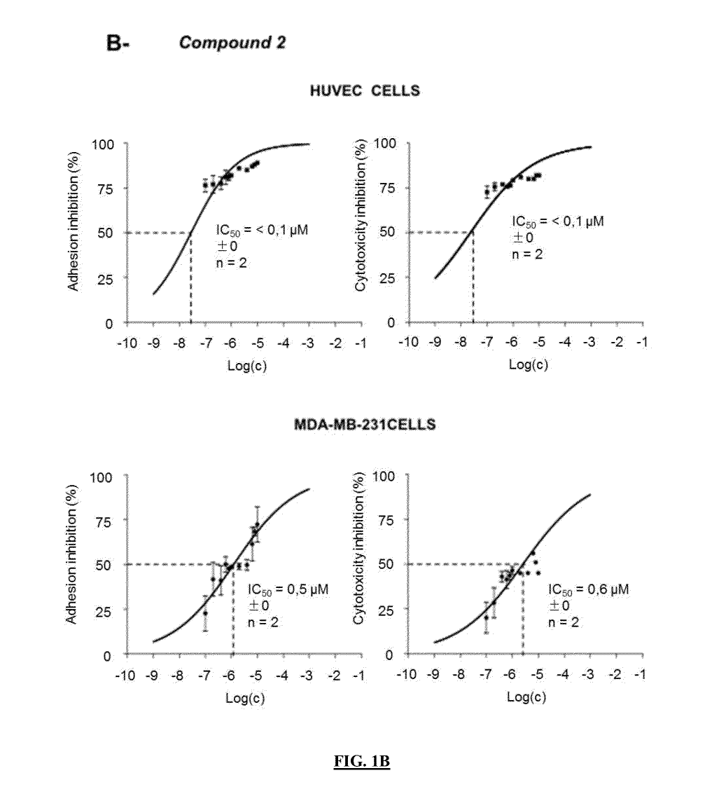

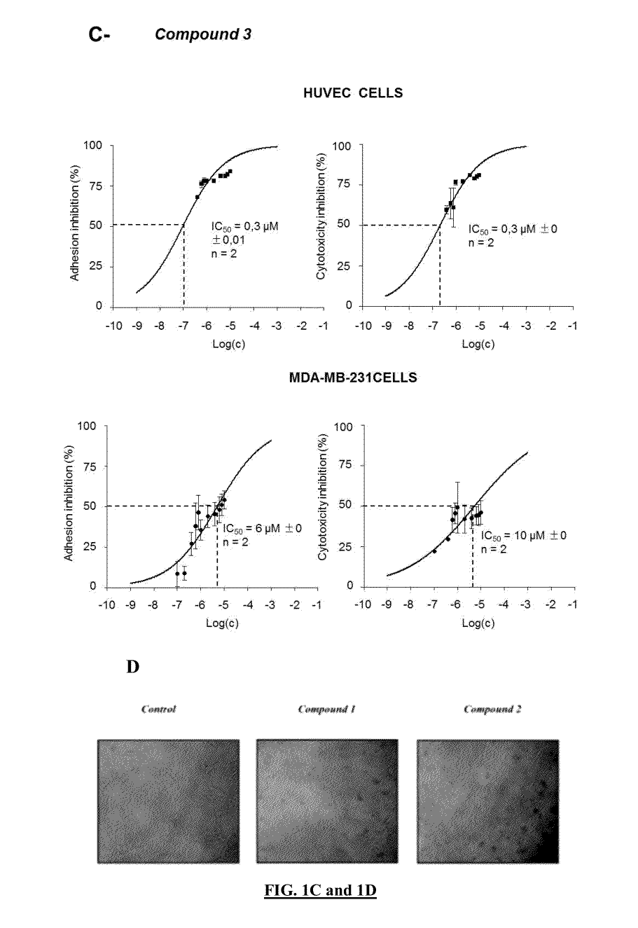

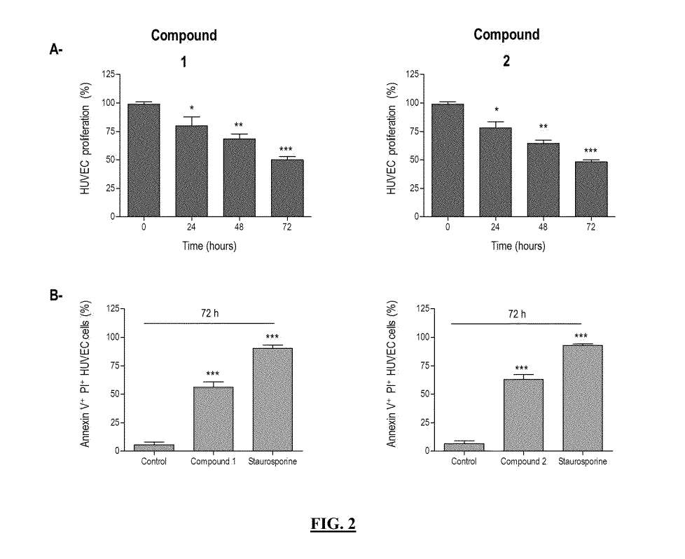

Moreover, these compounds also inhibited the adhesion of HUVEC cells (FIGS. 1A, B and C). Moreover, the presented results also showed that the anti-proliferative activity of the compounds 1 and 2 of the invention on endothelial cells is associated with the induction of apoptosis (FIG. 2).

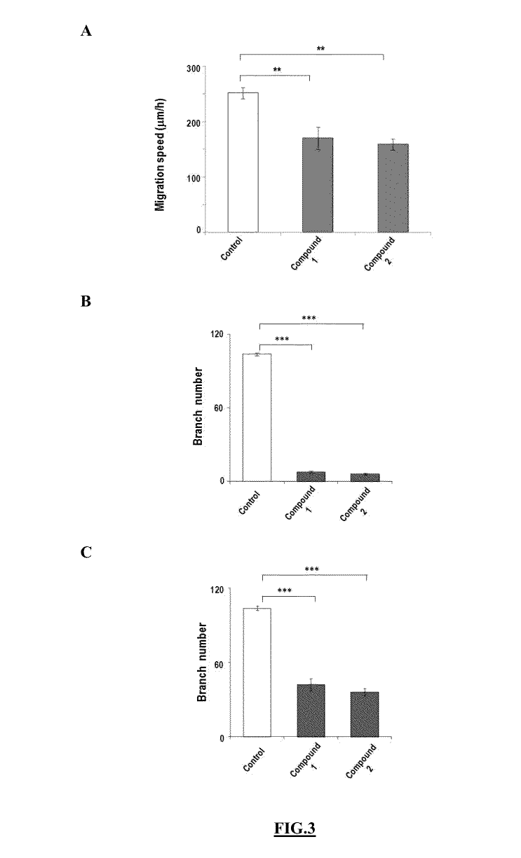

It could therefore be hypothesized that the compounds of the invention presented an anti-angiogenic effect. To confirm this hypothesis, an in vitro experiment for assessing the formation of blood vessels by endothelial cells in culture was carried out. First, we investigated the effect of the compounds of the invention on the migration speed of endothelial cells (FIG. 3A). Second, we analyzed the effect of the compounds of the invention on tube formation (FIGS. 1D and 3B), and on tube maintenance (FIG. 3C).

FIGS. 1D and 3 show that the compounds 1 and 2 of the invention effectively inhibited the formation and maintenance of new blood vessels.

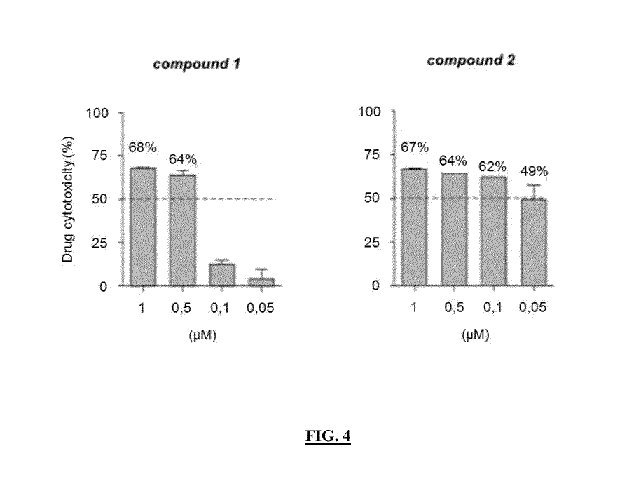

FIGS. 1A, B and C also showed that the compounds 1, 2 and 3 of the invention are cytotoxic for cancer cells in vitro, and that these compounds inhibited the adhesion of cancer cells in culture. The cytotoxicity of the compounds 1 and 2 of the invention was confirmed for different cancer cell lines. The effect of the compounds was assessed using wst-1 cytotoxic test (see for example FIG. 4). In conclusion, the compounds 1 and 2 of the invention are cytotoxic for all the tested cancer cell lines, whatever their origin (breast, colon, pancreas, cervical, prostate or brain cancer or osteosarcoma), as shown in the Tables 2 and 3 below.

TABLE 2

| Celllines | | | | % of Inhibition | |
|---|---|---|---|---|---|
| Type | Name | Concentration | | Compound 1 | Compound 2 |
| Breast | MDA-MB-438 | 1 | μM | 55% | 58% |
| Cancer | | 0.5 | μM | 40% | 51% |
| | | 0.1 | μM | 4% | 34% |
| | | 0.05 | μM | 2% | 13% |
| Colon | HCT-116 | 1 | μM | 56% | 60% |
| Cancer | | 0.5 | μM | 51% | 58% |
| | | 0.1 | μM | 7% | 49% |
| | | 0.05 | μM | 0% | 25% |
| Pancreas | PANC - 10.5 | 1 | μM | 56% | 65% |
| Cancer | | 0.5 | μM | 44% | 62% |
| | | 0.1 | μM | 21% | 51% |
| | | 0.05 | μM | 18% | 35% |
| Cervical | HeLa | 1 | μM | 68% | 67% |
| Cancer | | 0.5 | μM | 64% | 64% |
| | | 0.1 | μM | 13% | 62% |
| | | 0.05 | μM | 4% | 49% |
| Osteo- | U2OS | 1 | μM | 36% | 44% |
| sarcoma | | 0.5 | μM | 29% | 41% |
| | | 0.1 | μM | 15% | 31% |
| | | 0.05 | μM | 9% | 18% |
| Prostate | PC-3 | 1 | μM | 47% | 46% |
| Cancer | | 0.5 | μM | 25% | 46% |
| | | 0.1 | μM | 3% | 29% |
| | | 0.05 | μM | 0% | 0% |
| Brain | U87 | 1 | μM | 54% | 61% |
| Cancer | glioblastoma | 0.5 | μM | 45% | 60% |
| | | 0.1 | μM | 0% | 44% |
| | | 0.05 | μM | 0% | 19% |

TABLE 3

| | | Anti-proliferative activity | |
|---|---|---|---|
| | | IC₅₀ (μM) ± SD | |
| Human cancer celllines | | Compound 1 | Compound 2 |
| Breast | MDA-MB-231 | 0.6 ± 0.03 | 0.4 ± 0.02 |
| | MDA-MB-436 | 1.5 ± 0.02 | 2.3 ± 0.03 |
| | MDA-MB-438 | 1 ± 0.02 | 0.5 ± 0.03 |
| | BT-474 | 2 ± 0.05 | 1.6 ± 0.06 |
| Brain | U87 | 1 ± 0.01 | 0.3 ± 0.02 |
| | SH-SY5Y | 0.4 ± 0.03 | 0.05 ± 0.04 |
| Cervical | HeLa | 0.4 ± 0.04 | 0.05 ± 0.04 |
| Colon | HCT-116 | 1 ± 0.10 | 1 ± 0.02 |
| | HCT-29 | 0.5 ± 0.05 | 0.1 ± 0.01 |
| Fibrosarcoma | HT-1080 | 0.3 ± 0.23 | 0.07 ± 0.03 |
| Lung | A549 | 0.4 ± 0.04 | 0.1 ± 0.04 |
| | NCI-H460 | 0.5 ± 0.09 | 0.3 ± 0.04 |
| Osteosarcoma | U2OS | 2.3 ± 0.04 | 1.8 ± 0.05 |
| Pancreas | MIA PaCa-2 | 1 ± 0.01 | 1 ± 0.05 |
| | Panc-10.5 | 1 ± 0.09 | 0.1 ± 0.09 |
| Prostate | PC-3 | 1.7 ± 0.04 | 1.5 ± 0.12 |
| | LNCaP | 1.4 ± 0.13 | 1.3 ± 0.15 |
| Acute | CEM | 0.5 ± 0.01 | 0.3 ± 0.04 |
| lymphoblasticleukemia | | | |
| Burkitt'slymphoma | DAUDI | 1 ± 0.01 | 0.5 ± 0.02 |
| | RAJI | 1.5 ± 0.01 | 1.3 ± 0.04 |
| B lymphoma | VAL | 1.8 ± 0.04 | 1 ± 0.04 |
| Mantlecelllymphoma | GRANTA-519 | 1 ± 0.10 | 0.5 ± 0.02 |
| Myeloma | U266 | 2 ± 0.02 | 1.5 ± 0.04 |
| | OPM-2 | 2.5 ± 0.09 | 0.5 ± 0.01 |
| | KMS-II | 2.3 ± 0.01 | 1.8 ± 0.02 |
| Erythroleukemia | ERY-1 | 0.3 ± 0.04 | 0.1 ± 0.05 |

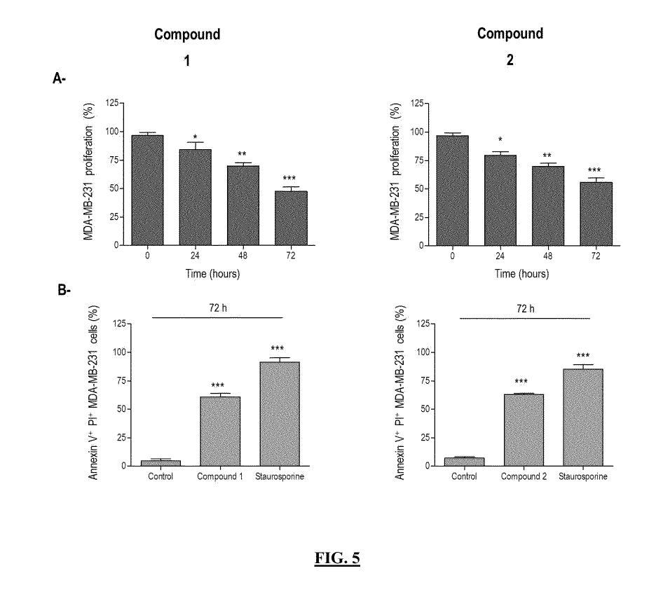

Moreover, as for endothelial cells, the anti-proliferative activity of the compounds 1 and 2 of the invention on cancer cells is associated with the induction of apoptosis (FIG. 5).

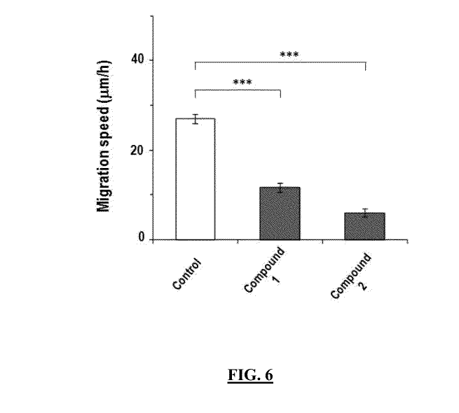

Furthermore, we analyzed the effect of the compounds of the invention on the motility of cancer cells, an essential step for invasion and metastasis. FIG. 6 shows that compounds 1 and 2 have a significant anti-migratory effect.

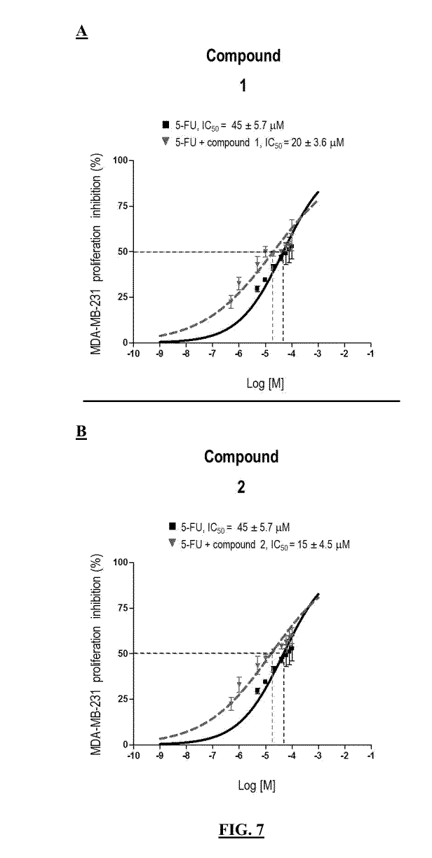

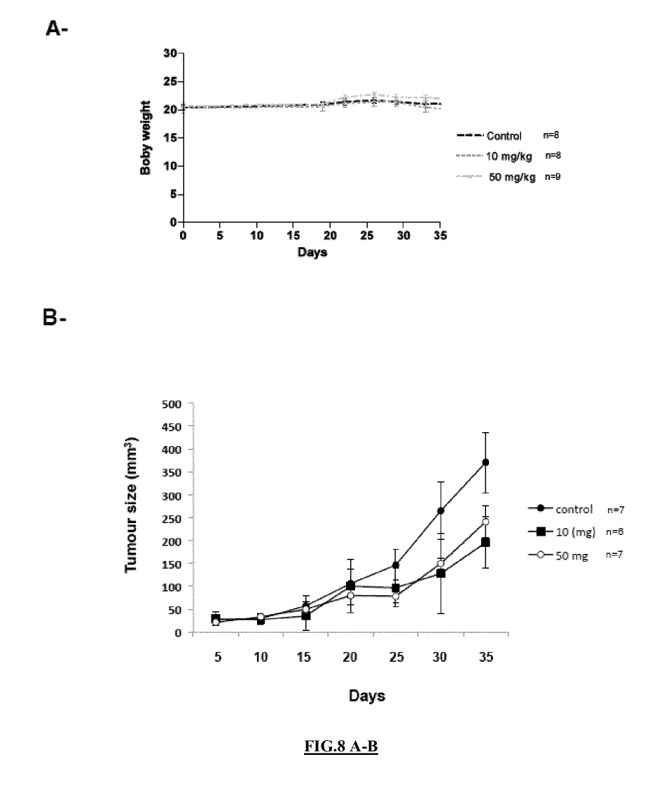

Finally, we tested the effect of the compounds of the invention in association with 5-FU, a chemotherapeutic agent on the proliferation of cancer cells (breast cancer cells, MDA-MB-231). As shown on FIG. 7, compounds 1 and 2 both increase the anti-proliferative effect of 5-FU on cancer cells after 72 hours of treatment.

Taken together, these results indicated an effect of the compounds 1 and 2 of the invention against cancer cells in vitro.

Furthermore, the potential in vivo anti-cancer effect of these compounds was tested in mice. NOD-SCID mice were injected with tumor, and then fed with the compounds of the invention.

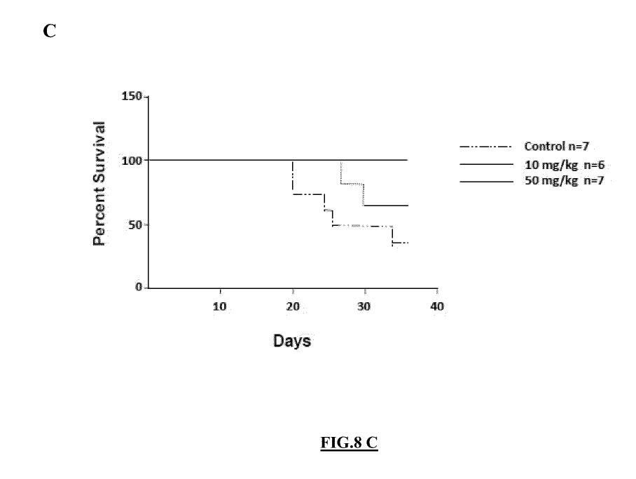

The results showed no significant change in body weight, demonstrating the absence of toxicity of the compound of the invention at a dose of 10 mg/kg as well as at a dose of 50 mg/kg (FIG. 8A).

The compound 1 of the invention inhibited the development of the tumor. Indeed, it is clear on FIG. 8B that the tumor size was smaller in mice fed with 10 mg/kg or 50 mg/kg of the compound 1.

Moreover, the mice fed with the compound 1 of the invention presented a percentage of survival at day 35 of 100% for the 50 mg/kg dose and about 70% for the 10 mg/kg dose, compared to less that 50% for the control (FIG. 8C).

Taken together, these results indicated an anti-cancer effect of the compound 1 of the invention in vivo.

The inventors showed that the compounds of the invention inhibited the binding of VEGF on its receptor Neuropilin-1 and on its receptor Neuropilin-2 (NRP-1 and NRP-2 respectively, see Tables 4 and 5 below), and, to a lesser extent, the binding of VEGF on its receptor VEGF-R1 (see Table 6 below).

TABLE 4

| Compound of | % VEGF/NRP-1 |
|---|---|
| the invention | binding inhibition |
| Compound 1 | 35 ± 4 |
| Compound 2 | 24 ± 2 |
| Compound 3 | 46 ± 3 |

TABLE 5

| | Inhibition of VEGF | Inhibition of VEGF |
|---|---|---|
| Compound of | binding to NRP-1 | binding to NRP-2 |
| the invention | IC₅₀ (μM) | IC₅₀ (μM) |
| Compound 1 | 38 ± 2.08 | 42 ± 3.15 |
| Compound 2 | 34 ± 3.11 | 35 ± 5.23 |

TABLE 6

| Compound of | % VEGF/VEGF-R1 |
|---|---|
| the invention | binding inhibition |
| Compound 1 | 18 ± 3 |
| Compound 2 | 7 ± 4 |
| Compound 3 | 7 ± 3 |

These results suggested that the compounds of the invention exerted their anti-angiogenic and anti-cancer effect through the inhibition of the pathway including VEGF, VEGF-R1, NRP-1 and NRP-2.

1. A compound of general formula (I), 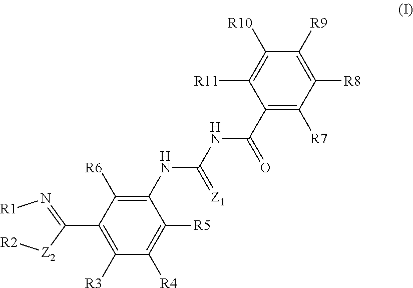
wherein
Z1 is S or O, preferably S;
—Z2- is —NR^{a}—, wherein R^{a} is H, OH, alkyl, alkoxy, aryl, alkenyl, alkynyl, amine, preferably R^{a} is H; —N═; O; S; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—; preferably —Z2- is —NH— or —N═;
each of R1 and R² is independently H, OH, alkyl, alkoxy, aryl, alkenyl, alkynyl, amine, NO₂, Cl, Br, I, F; preferably R1 and R2 together and with N and Z2 form an heterocycle eventually substituted, provided that said heterocycle is not an indole;
R5 is H, OH, C₁₋₄ alkyl, C₁₋₄ alkoxy, C₁₋₄alkenyl, C₁₋₄alkynyl, amine, preferably R5 is NH₂, NO₂, Cl, F, Br, I; more preferably R5 is H or CH₃; even more preferably R5 is CH₃;
each of R8 and R9 is independently, H, OH, alkyl, alkoxy, aryl, alkenyl, alkynyl, amine, NO₂, Cl, Br, I, F; preferably R₈ and R₉ together form a cycle comprising 5 or 6 atoms, preferably an heterocycle comprising 5 or 6 atoms, more preferably a 1,3-dioxacyclopentene or a 1,4-dioxane; and
each of R3, R4, R6, R7, R10 and R11 is independently, H, OH, C₁₋₄ alkyl, C₁₋₄ alkoxy, C₁₋₄alkenyl, C₁₋₄alkynyl, amine, NO₂, F, Cl, Br, I; preferably H, CH₃, OCH₃, OH, NH₂, NO₂, Cl, F, Br, I; more preferably is H or CH₃; even more preferably is H.

2. The compound according to claim 1, wherein
Z1 is S or O, preferably S;
—Z2- is —NR—, wherein R^{a} is H, OH, alkyl, alkoxy, aryl, alkenyl, alkynyl, amine, preferably H; —N═; O; S; —CR^{b}═, wherein R^{b} is H, OH, alkyl, alkoxy, aryl, alkenyl, alkynyl, amine, NO₂, Cl, Br, I, F, preferably H; —CHR^{c}—, wherein R^{c} is H, OH, alkyl, alkoxy, aryl, alkenyl, alkynyl, amine, NO₂, Cl, Br, I, F, or —CH₂—, preferably —Z2- is —NH— or —N
R1 and R2 together and with N and Z2 form an heterocycle eventually substituted, provided that said heterocycle is not an indole;
R⁵ is H or C₁₋₄ alkyl;
R8 and R9 together form a cycle comprising 5 or 6 atoms, preferably an heterocycle comprising 5 or 6 atoms, more preferably a 1,3-dioxacyclopentene or a 1,4-dioxane;
R3, R4, R6, R7, R10 and R11 are H or C₁₋₄ alkyl.

3. The compound according to claim 1, having general formula (Ia): 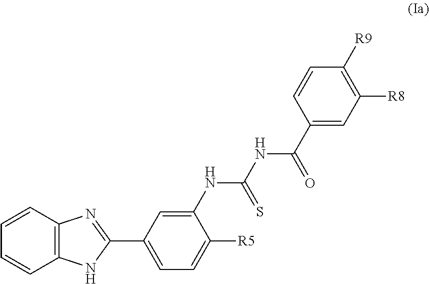
wherein
R5 is H or CH₃;
R8 and R9 together form a 1,3-dioxacyclopentene or a 1,4-dioxane.

4. The compound according to claim 1, being:
N-[5-(1H-benzimidazol-2-yl)-2-methylphenyl]-N′-(2,3-dihydro-1,4-benzodioxin-6-ylcarbonyl)thiourea 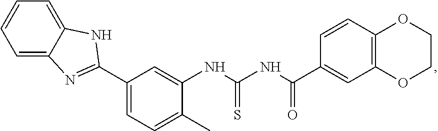 [SMILES: Cc1ccc(-c2nc3ccccc3[nH]2)cc1NC(=S)NC(=O)c1ccc2c(c1)OCCO2]
N-[3-(1H-benzimidazol-2-yl)phenyl]-N′-(2,3-dihydro-1,4-benzodioxin-6-ylcarbonyl)thiourea: 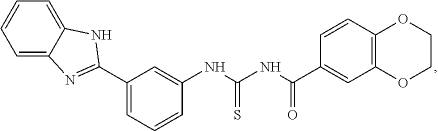 [SMILES: O=C(NC(=S)Nc1cccc(-c2nc3ccccc3[nH]2)c1)c1ccc2c(c1)OCCO2]
and/or
N-[3-(1H-benzimidazol-2-yl)phenyl]-N′-(1,3-benzodioxol-5-ylcarbonyl)thiourea

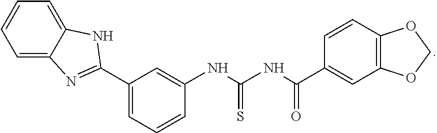 [SMILES: O=C(NC(=S)Nc1cccc(-c2nc3ccccc3[nH]2)c1)c1ccc2c(c1)OCO2]

5. A pharmaceutical composition comprising a compound according to claim 1, or salts, esters or mixtures thereof, in association with at least one pharmaceutically acceptable vehicle.

6. A medicament comprising a compound according to claim 1, or salts, esters or mixtures thereof.

7. A method for manufacturing a compound according to claim 1, where a compound of general formula (II) 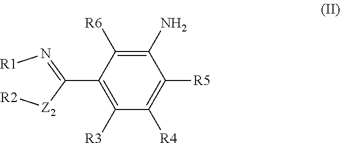
wherein R1, R2, Z2, R3, R4, R5 and R6 are as described in claim 1
is reacted with a compound of general formula (IV) 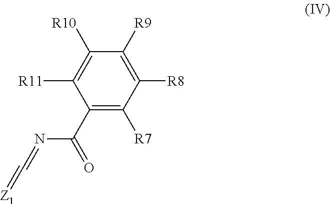
in a refluxing solvent, to give compounds of general formula (I).

8. The method according to claim 7, wherein the compound of general formula (IV) is obtained by reacting a compound of general formula (III) 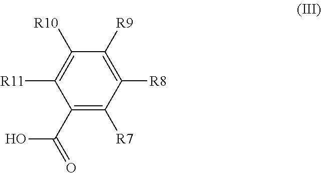
with (i) SOCl₂ and (ii) NH₄Z1CN, wherein Z1 is as defined in claim 1, in the presence of a base.

9. A method for inhibiting the Neuropilin pathway in a subject in need thereof, comprising administering to the subject a therapeutically effective amount of a compound according to claim 1.

10. A method for treating cancer in a subject in need thereof, comprising administering to the subject a therapeutically effective amount of a compound according to claim 1.

11. The method according to claim 10, wherein the method comprises for inhibiting the proliferation of cancer cells and/or inducing apoptosis of cancer cells, and/or inhibiting the growth of a tumor and/or inhibiting the number and the development of cancerous cells within the body of the subject and/or inhibiting the occurrence of metastases.

12. The method according to claim 10, wherein the cancer is one of carcinoma, adenocarcinoma, lymphoma, blastoma, hepatoma, sarcoma; leukemia, such as, for example, squamous cell cancer; lung cancer, including small-cell lung cancer, non-small cell lung cancer, adenocarcinoma of the lung, and squamous carcinoma of the lung; cancer of the peritoneum; hepatocellular cancer; gastric or stomach cancer, including gastrointestinal cancer; pancreatic cancer, such as, for example, pancreatic carcinoma; glioblastoma; neuroblastoma; cervical cancer; ovarian cancer; liver cancer; bladder cancer; hepatoma; breast cancer; colon cancer, such as, for example, colon adenocarcinoma; colorectal cancer, such as, for example, colorectal carcinoma; endometrial or uterine carcinoma; salivary gland carcinoma; osteosarcoma; kidney or renal cancer; liver cancer; prostate cancer, such as, for example, prostate adenocarcinoma; vulval cancer; thyroid cancer; hepatic carcinoma and various types of head and neck cancer; B-cell lymphoma, including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, chronic lymphocytic leukemia (CLL), acute lymphoblastic leukemia (ALL), Hairy cell leukemia, chronic myeloblastic leukemia; and post-transplant lymphoproliferative disorder (PTLD), abnormal vascular proliferation associated with phakomatoses, edema, such as, for example that associated with brain tumors; and Meigs' syndrome.

13. The method according to claim 10, wherein said subject is diagnosed with a cancer and/or underwent a surgical operation to remove a tumor and/or was previously treated with an anti-cancer treatment, and/or is simultaneously treated with an anti-cancer treatment, and/or is planned to be treated with an anti-cancer treatment, wherein the anti-cancer treatment may be chemotherapy and/or radiotherapy.

14. A method for treating a disease, disorder or condition related to angiogenesis in a subject in need thereof, comprising administering to the subject a therapeutically effective amount of a compound according to claim 1.

15. The method according to claim 14, wherein the method comprises inhibiting the formation of neovessels and/or the adhesion of endothelial cells and/or the growth, development or division of endothelial cells, and/or inducing apoptosis of endothelial cells in the subject.

16. The method according to claim 14, wherein the disease related to angiogenesis is an ophthalmic disease, a disease of the renal and/or urogenital tract; a disease of the digestive tract; a disease of the respiratory tract; a disease of bones or joints; a skin disease; an autoimmune disease; is consecutive to a graft; is a post-surgery reaction, a hyperproliferation or a hypertrophy; abnormal wound healing; chronic inflammation; a disease of the cardiovascular system or of blood or an infection.
